SEQUENCE LISTING (134 sequences)

SEQ ID NO: 1 - DNA, 1138 bp, Homo sapiens
taggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgctctttatcagatacaaaatttacgaacgtttgggataaaaagggagtcttaaagaaatgtaagatgtgctgggactacttagcctccaattcacagatacctggatggagcttatctttcttac

SEQ ID NO: 2 - DNA, 875 bp, Homo sapiens
tagccagatattccctgcggggcccgagagtcttccctatcagaccccgggatagggatgaggcccacagtcacccaccagactctttgtatagccccgttaagtgcaccccggcctggagggggtggttctgggtagaagcacgtccgggccgcgccggatgcctcctggaaggcgcctggacccacgccaggtttcccagtttaattcctcatgacttagcgtcccagcccgcgcaccgaccagcgccccagttccccacagacgccggcgggcccgggagcctcgcggacgtgacgccgcgggcggaagtgacgttttcccgcggttggacgcggcgctcagttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggtgagcgagcagagccgccgtcgcctcacgcgggaagggcgccccgggtgtgcgtagggcgggcgccaaggcggctcggcggggatccgtcctcgccaggggccgggtcccggcgggaggaggcgccctccctgcccccgccacggcggagcgtctgcagaatggtgacaggattctgggttcttgggcgaggggtctcggcttcaacttgacaggtgtcgggcgggtggggc

SEQ ID NO: 3 - DNA, 596 bp, Homo sapiens
taggggaaccaaactctgtttccaggggagtggagagagaaactgggtccccctcccgtagctcctgggacacagctgagccagccacaggatctggggacaaccggggcggatcccccctttcgggaggcggtggcatcagttcagagtccgcatttttattcatcggggaagcgtggggagaaggatgggctggagctgggtcctggtctgaaggacagcagtccggagctaacggttgagtctccaaagtcttcatactgcagaggaagcacagcggagattagcctcagccaggatggcttcgaagttctcagggatccgacgcagagctaaagaaacccacctgtgcttccctcctcttctgggagtaggcagaagactcccgggaggagaggcgaacagcggacgccaattcttttgaaagcactgtgttccttagcaccgcgggtcgctacgggcctcttgctgtcgcgggatttcggtccaccttccgattgggccgccgcatcccggatcagatttcgcgggcgacccacggaacccgcggagccgggacgtgaaaggttagaaggtttcccgttcccatcaagccc

SEQ ID NO: 4 - DNA, 831 bp, Homo sapiens
tagggctcctcgtggctgctgggagttgtagtctgaacgcttctatcttggcgagaagcgcctacgctccccctaccgagtcccgcggtaattcttaaagcacctgcaccgcccccccgccgcctgcagagggcgcagcaggtcttgcacctcttctgcatctcattctccaggcttcagacctgtctccctcattcaaaaaatatttattatcgagctcttacttgctacccagcactgatataggcactcaggaatacaacaatgaataagatagtagaaaaattctatatcctcataaggcttacgtttccatgtactgaaagcaatgaacaaataaatcttatcagagtgataagggttgtgaaggagattaaataagatggtgtgatataaagtatctgggagaaaacgttagggtgtgatattacggaaagccttcctaaaaaatgacattttaactgatgagaagaaaggatccagctgagagcaaacgcaaaagctttcttccttccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttgatgggattggggttttcccctcccatgtgctcaagactggcgctaaaagttttgagcttctcaaaagtc

SEQ ID NO: 5 - DNA, 623 bp, Homo sapiens
tagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgcgcaggttcttggtgaccctccggattcggcgcgcgtgcggcccgccgcgagtgagggttttcgtggttcacatcccgcggctcacgggggagtgggcagcgccaggggcgcccgccgctgtggccctcgtgctgatgctactgaggagccagcgtc

SEQ ID NO: 6 - DNA, 675 bp, Homo sapiens
tagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggc

SEQ ID NO: 7 - DNA, 185 bp, Homo sapiens
taggaaggagagagtgcgccggagcagcgtgggaaagaagggaagagtgtcgttaagtttacggccaacggtggattatccgggccgctgcgcgtctgggggctgcggaatgcgcgaggagaacaagggcatgcccagtgggggcggcagcgatgagggtctggccagcgccgcggcgcggggac

SEQ ID NO: 8 - DNA, 450 bp, Homo sapiens
tagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccc

SEQ ID NO: 9 - DNA, 1315 bp, Homo sapiens
tagtctccggcgctggcgcctatggtcggcctccgacagcgctccggagggaccgggggagctcccaggcgcccgggtgagtagccaggcgcggctccccggtccccccgacccccggcgccagcttttgctttcccagccagggcgcggtggggtttgtccgggcagtgcctcgagcaactgggaaggccaaggcggagggaaacttggcttcggggagaagtgcgatcgcagccgggaggcttccccagccccgcgggccgggtgagaacaggtggcgccggcccgaccaggcgctttgtgtcggggcgcgaggatctggagcgaactgctgcgcctcggtgggccgctcccttccctcccttgctcccccgggcggccgcacgccgggtcggccgggtaacggagagggagtcgccaggaatgtggctctggggactgcctcgctcggggaaggggagagggtggccacggtgttaggagaggcgcgggagccgagaggtggcgcgggggtgccaccgttgccgcaggctggagagagattgctcccagtgaggcgcgtaccgtctgggcgagggcttcattcttccgcggcgtccctggaggtgggaaagctgggtgggcatgtgtgcagagaaaggggaggcggggaggccagtcacttccggagccggttctgatcccaacagaccgcccagcgtttggggacgccgacctcggggtgccgtggtgcccggccccacgcgcgcgcggggctgaggggtcgggggcgtccctggccgcccagctttaacaaagggtgctcctctccaccccgcgaggaggggcagctccggagacccggtcttcagcgagcggggtcttagcgccggggaggtctacttccttttggggttgccattttactattattattgccttttttttttcttcaaaaggactggagactgatgcatgagggggctacggaggcgcaggagcggtggtgatggtctgggaagcggagctgaagtgccctgggctttggtgaggcgtgacagtttatcatgaccgtgttcaggcaggaaaacgtggatgattactacgacaccggcgaggaacttggcaggtaaagggggtaccagaagcgtaccctcctggattgtggaaatgcataacgatggggccattgggtggtaaacaaatgcagtttgaatcaggcgtctccctcgccctttctggagatgcgcaaatcatagagaaaagagttactaacccagcggtaaaccgcctgatccaagggcctgggggtggaggagaggcagcagttcagggc

SEQ ID NO: 10 - DNA, 294 bp, Homo sapiens
tagcacagtaaagctggcctccagaaacacgggtatctccgcgtggtgctttgcggtcgccgtcgttgtggccgtccggggtggggtgtgaggaggggacgaaggagggaaggaagggcaaggcggggggggctctgcgagagcgcgcccagccccgccttcgggccccacagtccctgcacccaggtttccattgcgcggctctcctcagctccttcccgccgcccagtctggatcctgggggaggcgctgaagtcggggcccgccctgtggccccgcccggcccgcgcttgc

SEQ ID NO: 11 - DNA, 592 bp, Homo sapiens
tagttagaaaaagccaaagattgtgcgatttatgccccaaacccccttgtaaggggattctcacctcaacttgtcttctgtggtcagtgtttcccgcccctgaatcagggttactgtcactatggctttcaattggcccggcgtaggcgcatgctctgcgcgtattggcctccgctcctgtccccagacaagcggccatcttgggtcccgcccctaccgtggggtcttctgggaattgcagtccccgctctgctctgtccggtcacaggactttttgccctctgttcccgggtccctcaggcggccacccagtgggcacactcccaggcggcgctccggccccgcgctccctccctctgcctttcattcccagctgtcaacatcctggaaggtaggggcggggaggcaagcccaagtggaatactgtttctggggcgcgggtctgggtttccacgcgcgtcaggtcatcaccccggagcccagtggggccggcgccgctcacggggcggtgggcttctgtcccgagtactcttccgccccacgagggtctcagggtggggactcgggccccccaattcccaagcaccgaccc

SEQ ID NO: 12 - DNA, 729 bp, Homo sapiens
taggcaggctgtgcggttgggcggggccctgtgccccactgcggagtgcgggtcgggaagcggagagagaagcagctgtgtaatccgctggatgcggaccagggcgctccccattcccgtcgggagcccgccgattggctgggtgtgggcgcacgtgaccgacatgtggctgtattggtgcagcccgccagggtgtcactggagacagaatggaggtgctgccggactcggaaatggggtaggtgctggagccaccatggccaggcttgctgcggggggaggggggaaggtggttttccctcgcactgtcttaaaccgatggcctttccttggcacagggtccactgcagcatgccaaacgaggaggcaggggcgtcgtccccccgccccccactgcagcactggagatggatttcctgtacttcggatccagggtttttgacagaagaggaagaagggggaggggtagaagtgttaaggggagtctgctgagaaaagctgtttttgaagccagaaggggtttttgtttttataatgccatttgacagagtggaataacagtatctaaggaaacgggtagaggacaacaaagaatggagcatattcatggcgaggagcaaaagctctaccccattgaaaggcttcttttcctccctggcgacaaggacacatgcattggtggccaaaagagagaggagacaaaaccgctgcagatggctgatgtgaatc

SEQ ID NO: 13 - DNA, 22 bp, Artificial Sequence
  Designed biotinated oligonucleotide for fixation
atagggaaagtatggcttcttc

SEQ ID NO: 14 - DNA, 22 bp, Artificial Sequence
  Designed biotinated oligonucleotide for fixation
aagtcatgaggaattaaactgg

SEQ ID NO: 15 - DNA, 22 bp, Artificial Sequence
  Designed biotinated oligonucleotide for fixation
gtgcttcctctgcagtatgaag

SEQ ID NO: 16 - DNA, 22 bp, Artificial Sequence
  Designed biotinated oligonucleotide for fixation
acccttatcactctgataagat

SEQ ID NO: 17 - DNA, 22 bp, Artificial Sequence
  Designed biotinated oligonucleotide for fixation
tggctcctcagtagcatcagca

SEQ ID NO: 18 - DNA, 22 bp, Artificial Sequence
  Designed biotinated oligonucleotide for fixation
cctcctttccagctgggagaca

SEQ ID NO: 19 - DNA, 22 bp, Artificial Sequence
  Designed biotinated oligonucleotide for fixation
cactgggcatgcccttgttctc

SEQ ID NO: 20 - DNA, 22 bp, Artificial Sequence
  Designed biotinated oligonucleotide for fixation
acctgtgacacattgctaagct

SEQ ID NO: 21 - DNA, 22 bp, Artificial Sequence
  Designed biotinated oligonucleotide for fixation
tgggagcaatctctctccagcc

SEQ ID NO: 22 - DNA, 22 bp, Artificial Sequence
  Designed biotinated oligonucleotide for fixation
aacctgggtgcagggactgtgg

SEQ ID NO: 23 - DNA, 22 bp, Artificial Sequence
  Designed biotinated oligonucleotide for fixation
acactgaccacagaagacaagt

SEQ ID NO: 24 - DNA, 22 bp, Artificial Sequence
  Designed biotinated oligonucleotide for fixation
cttagatactgttattccactc

SEQ ID NO: 25 - DNA, 30 bp, Artificial Sequence
  Designed oligonucleotide
ccaacgctggctctggcgagggctgcttcc

SEQ ID NO: 26 - DNA, 30 bp, Artificial Sequence
  Designed oligonucleotide
ggctggtgcccccgggggagacccaacctg

SEQ ID NO: 27 - DNA, 30 bp, Artificial Sequence
  Designed oligonucleotide
gggcgacttcaggggtgccacattcgctaa

SEQ ID NO: 28 - DNA, 30 bp, Artificial Sequence
  Designed oligonucleotide
gtgctcggagttaatagcacctcctccgag

SEQ ID NO: 29 - DNA, 30 bp, Artificial Sequence
  Designed oligonucleotide
cactcgctcacggcgtccccttgcctggaa

SEQ ID NO: 30 - DNA, 30 bp, Artificial Sequence
  Designed oligonucleotide
agataccgcggtccctccagaggatttgag

SEQ ID NO: 31 - DNA, 30 bp, Artificial Sequence
  Designed oligonucleotide
ggacagggtcggagggggctcttccgccag

SEQ ID NO: 32 - DNA, 30 bp, Artificial Sequence
  Designed oligonucleotide
caccggaggaagaaagaggaggggctggct

SEQ ID NO: 33 - DNA, 30 bp, Artificial Sequence
  Designed oligonucleotide
ggtcaccagagggtggggcggaccgcgtgc

SEQ ID NO: 34 - DNA, 30 bp, Artificial Sequence
  Designed oligonucleotide
gctcggcggctgcggagagggggagagcag

SEQ ID NO: 35 - DNA, 30 bp, Artificial Sequence
  Designed oligonucleotide
gcagcgggcggcggggagcagcatggagcc

SEQ ID NO: 36 - DNA, 30 bp, Artificial Sequence
  Designed oligonucleotide
ggcggcggggagcagcatggagccttcggc

SEQ ID NO: 37 - DNA, 30 bp, Artificial Sequence
  Designed oligonucleotide
tgactggctggccacggccgcggcccgggg

SEQ ID NO: 38 - DNA, 30 bp, Artificial Sequence
  Designed oligonucleotide
tcgggtagaggaggtgcgggcgctgctgga

SEQ ID NO: 39 - DNA, 30 bp, Artificial Sequence
  Designed oligonucleotide
ggcgggggcgctgcccaacgcaccgaatag

SEQ ID NO: 40 - DNA, 30 bp, Artificial Sequence
  Designed oligonucleotide
ttacggtcggaggccgatccaggtgggtag

SEQ ID NO: 41 - DNA, 30 bp, Artificial Sequence
  Designed oligonucleotide
agggtctgcagcgggagcaggggatggcgg

SEQ ID NO: 42 - DNA, 30 bp, Artificial Sequence
  Designed oligonucleotide
gcgactctggaggacgaagtttgcagggga

SEQ ID NO: 43 - DNA, 30 bp, Artificial Sequence
  Designed oligonucleotide
cacagacgccggcgggcccgggagcctcgc

SEQ ID NO: 44 - DNA, 30 bp, Artificial Sequence
  Designed oligonucleotide
ggacgtgacgccgcgggcggaagtgacgtt

SEQ ID NO: 45 - DNA, 30 bp, Artificial Sequence
  Designed oligonucleotide
ttcccgcggttggacgcggcgctcagttgc

SEQ ID NO: 46 - DNA, 30 bp, Artificial Sequence
  Designed oligonucleotide
cgggcgggggagggcgcgtccggtttttct

SEQ ID NO: 47 - DNA, 30 bp, Artificial Sequence
  Designed oligonucleotide
caggggacgttgaaattatttttgtaacgg

SEQ ID NO: 48 - DNA, 30 bp, Artificial Sequence
  Designed oligonucleotide
gagtcgggagaggacggggcgtgccccgac

SEQ ID NO: 49 - DNA, 30 bp, Artificial Sequence
  Designed oligonucleotide
gtgcgcgcgcgtcgtcctccccggcgctcc

SEQ ID NO: 50 - DNA, 30 bp, Artificial Sequence
  Designed oligonucleotide
tccacagctcgctggctcccgccgcggaaa

SEQ ID NO: 51 - DNA, 30 bp, Artificial Sequence
  Designed oligonucleotide
ggcgtcatgccgcccaaaaccccccgaaaa

SEQ ID NO: 52 - DNA, 30 bp, Artificial Sequence
  Designed oligonucleotide
acggccgccaccgccgccgctgccgccgcg

SEQ ID NO: 53 - DNA, 33 bp, Artificial Sequence
  Designed oligonucleotide
gaacccccggcaccgccgccgccgccccctcct

SEQ ID NO: 54 - DNA, 30 bp, Artificial Sequence
  Designed oligonucleotide
ggacagcggcccggaggacctgcctctcgt

SEQ ID NO: 55 - DNA, 30 bp, Artificial Sequence
  Designed oligonucleotide
caggtgagcgagcagagccgccgtcgcctc

SEQ ID NO: 56 - DNA, 30 bp, Artificial Sequence
  Designed oligonucleotide
acgcgggaagggcgccccgggtgtgcgtag

SEQ ID NO: 57 - DNA, 30 bp, Artificial Sequence
  Designed oligonucleotide
ggcgggcgccaaggcggctcggcggggatc

SEQ ID NO: 58 - DNA, 30 bp, Artificial Sequence
  Designed oligonucleotide
cgtcctcgccaggggccgggtcccggcggg

SEQ ID NO: 59 - DNA, 30 bp, Artificial Sequence
  Designed oligonucleotide
aggaggcgccctccctgccccccgccacgg

SEQ ID NO: 60 - DNA, 30 bp, Artificial Sequence
  Designed oligonucleotide
cggagcgtctgcagaatggtgacaggattc

SEQ ID NO: 61 - DNA, 34 bp, Artificial Sequence
  Designed oligonucleotide
ctcccgggaggagaggcgaacagcggacgccaat

SEQ ID NO: 62 - DNA, 30 bp, Artificial Sequence
  Designed oligonucleotide
ttagcaccgcgggtcgctacgggcctcttg

SEQ ID NO: 63 - DNA, 30 bp, Artificial Sequence
  Designed oligonucleotide
ctgtcgcgggatttcggtccaccttccgat

SEQ ID NO: 64 - DNA, 30 bp, Artificial Sequence
  Designed oligonucleotide
tgggccgccgcatcccggatcagatttcgc

SEQ ID NO: 65 - DNA, 30 bp, Artificial Sequence
  Designed oligonucleotide
gggcgacccacggaacccgcggagccggga

SEQ ID NO: 66 - DNA, 30 bp, Artificial Sequence
  Designed oligonucleotide
cgtgaaaggttagaaggtttcccgttccca

SEQ ID NO: 67 - DNA, 30 bp, Artificial Sequence
  Designed oligonucleotide
ctgaacgcttctatcttggcgagaagcgcc

SEQ ID NO: 68 - DNA, 30 bp, Artificial Sequence
  Designed oligonucleotide
tacgctccccctaccgagtcccgcggtaat

SEQ ID NO: 69 - DNA, 30 bp, Artificial Sequence
  Designed oligonucleotide
tcttaaagcacctgcaccgcccccccgccg

SEQ ID NO: 70 - DNA, 30 bp, Artificial Sequence
  Designed oligonucleotide
cctgcagagggcgcagcaggtcttgcacct

SEQ ID NO: 71 - DNA, 30 bp, Artificial Sequence
  Designed oligonucleotide
caaagggcggcgcagcggctgccgagctcg

SEQ ID NO: 72 - DNA, 30 bp, Artificial Sequence
  Designed oligonucleotide
gccctggaggcggcgagaacatggtgcgca

SEQ ID NO: 73 - DNA, 30 bp, Artificial Sequence
  Designed oligonucleotide
ggttcttggtgaccctccggattcggcgcg

SEQ ID NO: 74 - DNA, 30 bp, Artificial Sequence
  Designed oligonucleotide
cgtgcggcccgccgcgagtgagggttttcg

SEQ ID NO: 75 - DNA, 30 bp, Artificial Sequence
  Designed oligonucleotide
tggttcacatcccgcggctcacgggggagt

SEQ ID NO: 76 - DNA, 30 bp, Artificial Sequence
  Designed oligonucleotide
gggcagcgccaggggcgcccgccgctgtgg

SEQ ID NO: 77 - DNA, 30 bp, Artificial Sequence
  Designed oligonucleotide
agggggagaccggagagagaacgtacgccg

SEQ ID NO: 78 - DNA, 30 bp, Artificial Sequence
  Designed oligonucleotide
aggagtcaggcggcgggatcaaggggagtc

SEQ ID NO: 79 - DNA, 30 bp, Artificial Sequence
  Designed oligonucleotide
ggggtgtctgggcgcggggcagagcgtgga

SEQ ID NO: 80 - DNA, 30 bp, Artificial Sequence
  Designed oligonucleotide
ggcggcagcggccaacggtcgccaagacaa

SEQ ID NO: 81 - DNA, 30 bp, Artificial Sequence
  Designed oligonucleotide
ccattctacgcgaggacgcggcgacaggag

SEQ ID NO: 82 - DNA, 35 bp, Artificial Sequence
  Designed oligonucleotide
gggagcggccagcaggggaggggagcgcgggggaa

SEQ ID NO: 83 - DNA, 30 bp, Artificial Sequence
  Designed oligonucleotide
taggaaggagagagtgcgccggagcagcgt

SEQ ID NO: 84 - DNA, 30 bp, Artificial Sequence
  Designed oligonucleotide
gggaaagaagggaagagtgtcgttaagttt

SEQ ID NO: 85 - DNA, 30 bp, Artificial Sequence
  Designed oligonucleotide
acggccaacggtggattatccgggccgctg

SEQ ID NO: 86 - DNA, 30 bp, Artificial Sequence
  Designed oligonucleotide
cgcgtctgggggctgcggaatgcgcgagga

SEQ ID NO: 87 - DNA, 30 bp, Artificial Sequence
  Designed oligonucleotide
gaacaagggcatgcccagtgggggcggcag

SEQ ID NO: 88 - DNA, 35 bp, Artificial Sequence
  Designed oligonucleotide
cgatgagggtctggccagcgccgcggcgcggggac

SEQ ID NO: 89 - DNA, 33 bp, Artificial Sequence
  Designed oligonucleotide
ccccattggccgcctgccggccgccctccgccc

SEQ ID NO: 90 - DNA, 30 bp, Artificial Sequence
  Designed oligonucleotide
aaaaggcggcaaggagccgagaggctgctt

SEQ ID NO: 91 - DNA, 30 bp, Artificial Sequence
  Designed oligonucleotide
cggagtgtgaggaggacagccggaccgagc

SEQ ID NO: 92 - DNA, 30 bp, Artificial Sequence
  Designed oligonucleotide
caacgccggggactttgttccctccgcgga

SEQ ID NO: 93 - DNA, 30 bp, Artificial Sequence
  Designed oligonucleotide
ggggactcggcaactcgcagcggcagggtc

SEQ ID NO: 94 - DNA, 34 bp, Artificial Sequence
  Designed oligonucleotide
tggggccggcgcctgggagggatctgcgcccccc

SEQ ID NO: 95 - DNA, 35 bp, Artificial Sequence
  Designed oligonucleotide
tagtctccggcgctggcgcctatggtcggcctccg

SEQ ID NO: 96 - DNA, 30 bp, Artificial Sequence
  Designed oligonucleotide
acagcgctccggagggaccgggggagctcc

SEQ ID NO: 97 - DNA, 30 bp, Artificial Sequence
  Designed oligonucleotide
caggcgcccgggtgagtagccaggcgcggc

SEQ ID NO: 98 - DNA, 30 bp, Artificial Sequence
  Designed oligonucleotide
tccccggtccccccgacccccggcgccagc

SEQ ID NO: 99 - DNA, 30 bp, Artificial Sequence
  Designed oligonucleotide
ttttgctttcccagccagggcgcggtgggg

SEQ ID NO: 100 - DNA, 30 bp, Artificial Sequence
  Designed oligonucleotide
tttgtccgggcagtgcctcgagcaactggg

SEQ ID NO: 101 - DNA, 35 bp, Artificial Sequence
  Designed oligonucleotide
ctctgcgagagcgcgcccagccccgccttcgggcc

SEQ ID NO: 102 - DNA, 31 bp, Artificial Sequence
  Designed oligonucleotide
ttgcgcggctctcctcagctccttcccgccg

SEQ ID NO: 103 - DNA, 34 bp, Artificial Sequence
  Designed oligonucleotide
cccagtctggatcctgggggaggcgctgaagtcg

SEQ ID NO: 104 - DNA, 35 bp, Artificial Sequence
  Designed oligonucleotide
gggcccgccctgtggccccgcccggcccgcgcttg

SEQ ID NO: 105 - DNA, 30 bp, Artificial Sequence
  Designed oligonucleotide
tccagaaacacgggtatctccgcgtggtgc

SEQ ID NO: 106 - DNA, 33 bp, Artificial Sequence
  Designed oligonucleotide
tttgcggtcgccgtcgttgtggccgtccggggt

SEQ ID NO: 107 - DNA, 30 bp, Artificial Sequence
  Designed oligonucleotide
ccaagtggaatactgtttctggggcgcggg

SEQ ID NO: 108 - DNA, 30 bp, Artificial Sequence
  Designed oligonucleotide
tctgggtttccacgcgcgtcaggtcatcac

SEQ ID NO: 109 - DNA, 30 bp, Artificial Sequence
  Designed oligonucleotide
cccggagcccagtggggccggcgccgctca

SEQ ID NO: 110 - DNA, 30 bp, Artificial Sequence
  Designed oligonucleotide
cggggcggtgggcttctgtcccgagtactc

SEQ ID NO: 111 - DNA, 30 bp, Artificial Sequence
  Designed oligonucleotide
ttccgccccacgagggtctcagggtgggga

SEQ ID NO: 112 - DNA, 30 bp, Artificial Sequence
  Designed oligonucleotide
ctcgggccccccaattcccaagcaccgacc

SEQ ID NO: 113 - DNA, 30 bp, Artificial Sequence
  Designed oligonucleotide
gttgggcggggccctgtgccccactgcgga

SEQ ID NO: 114 - DNA, 30 bp, Artificial Sequence
  Designed oligonucleotide
gtgcgggtcgggaagcggagagagaagcag

SEQ ID NO: 115 - DNA, 30 bp, Artificial Sequence
  Designed oligonucleotide
ctgtgtaatccgctggatgcggaccagggc

SEQ ID NO: 116 - DNA, 30 bp, Artificial Sequence
  Designed oligonucleotide
gctccccattcccgtcgggagcccgccgat

SEQ ID NO: 117 - DNA, 30 bp, Artificial Sequence
  Designed oligonucleotide
tggctgggtgtgggcgcacgtgaccgacat

SEQ ID NO: 118 - DNA, 30 bp, Artificial Sequence
  Designed oligonucleotide
gtggctgtattggtgcagcccgccagggtg

SEQ ID NO: 119 - DNA, 2661 bp, Homo sapiens
acagacatgtgccaccatgcccagctaattttttgtttgtttgtttgtttgtttgtatttttagtagagatggggttttgccatgttggccaggctggtctcgaactcctgacctcgaatgataatgatccgccgcttggcctccaaagtgctaggattacaggtgtgagccactgcgccaggcctgggcactttctttagtagtttgaggagcaacatttttgacagtgtccttctgctcaagattcaagatcccagataaaattaaaccatctagagagatggcttgattggccaaacctggatctcatgaccacttcttgaagtgggtaagtctcataaatgctcagtccttccactatgcaactgagtggggtgggtgggaagcccctcaaaggaaaatccggttgttcttactagaaagaaaaggaaaatggatgtgaggcagtcaaaatcagcagaggtccaccacaccaccaaaatgtggtgattaaatatggagagacagagactaacagaggtatgtgaatattgaagtatgtctggacaatagcccaatgatgagaccaataaaatggttaccaaaatctggttttgagtagtagtgttaaatcagaccatttagtaaccattttttgttgcaaagtttctagcactgcccaaaccctgagtggtatatgaataactcgtccattatgtatctctttccagtcagcataatttatcccccacctatattcttttctgaccactcctacttccttctctttaccaaaatctaaactctaaggctgtttcttcagcaacttctttgtttagattggaagataaattaaacagcatgcgatgttttactgactttcagtatttaacagaggtgatttaatttttttttaaatccaaagtcaaacttctttataagatgaaggagaaaaatgtcttataaaatgcatatgtgaagatgccttctgagtgctttctcatgcagacttgttctagtctttaatgaatcttccttgtagacactgtggagatgaaagatggttctccacttctactcaaagtacaaatcaggccggcattttgaaaaagagacaggtttattcatagctgcagcgttagctggctttgttccctgtacaatttcacttttggttattaaaatattcactgtaggaaataaatttgtaacccatttctcatattacctacacacagaaaaacaaaatttgatatcctggggtttatttgctgagggcgcttcccataaaagcgagagagtgtgcgttgggaaatgtgtctggttaactcttttatggataaactttagtcacaatcctcccccgcccccctctcacccccagcaccctcccaacctcccgacttcccgcctctcaagggctggtgacctaatagcatttttcttcgtgcatattttggcgtcgccccatggcctggctgccttcgcctgtctgagttttttgaaattcctgcatgttcgccccagattaagccagtgtgtctcaggatgtgtgttccgttttgttctttccccttaacgctccctgtgcaacgtgtctggggggaggagggcagggacgggagagagggaggggcagaggcgaggagctgtccgccttgcacgtttccaatcgcattacgtgaacaaatagctgaggggcggccgggccagaacggcttgtgtaactttgcaaacgtgccagaaagtttaaatctctcctccttccttcactccagacactgcccgctctccgggactgccgcgcggctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacg

SEQ ID NO: 120 - DNA, 1953 bp, Homo sapiens
tataaattccacgcaggcattgaattgaatttgttcttaaccaaatgcgttttatctatacctggcaggaatctagaagtgaaattacaagatttatttcattttaattctattatgaagcatttaatcacaaataccctgaaaatgaaaagataatttatcattttaccttgactgagcaactctcctcacttcacattcatgaatccataacgcagagaggagactggatgattaagtgtttgattagagaaaacagattaacctagcaaacataataaatttggctcataagcaggatggctttataaatgctcacaatacctctcctgtataaaatcatgaaccacttcctacagtgatgactccatcgaaatagttgagaaacataaagcaaatgcatgtttatggctttctctttgagacattaaaagggtattgaaaggcatatctgattcagcttataactctggatatatattaaggaacatgtaagaaaatattaatgcataaaaaaagctacaacttctcaagtgttctagtttccactttgtcaataattacgttttcaatgtccttctgtggactgtttccaaaggtgccaatccagacccaaagtttcagatcactcagattcacccttaaccttcataacacaacccaatagctttacgaaaaaagttgcatatttaggtagttgttatcccattatgacaaaatacataaaattagcgagatattttttagccttcaaataagtgggaaaaaatccttttagctgagattccatttacatcagaataaaaatctaagttatgactaggttgaagcaacgtcctgtgcagcgctccataaagttcacttagtcttcaagggttccttacttagctaggttagtattcctggcctctttttttagcagtgagaaaaaggatactctccctgccccagctttatttttaaactcacagccatatcctggaggtctctgctggctatttggcgcgtgggggcggaggggggccgggggaggggggcggggcggggtctggaggtctgtgctggctatctggcgtgtgtgtgtgtgtgtgtgtgtgtgtgtgtggttggaggtctctgctggctatctggcgtgtgtgtgtgtgtggtgtggtgtgtgtaagcagtgaggttgttttagggccagtccttcctccgccactttgctgactcaaagacccagaggctttcttggggtgcaggtaccatgattccttgggccctaagggaatttttgttaggctagaagagtgggtgtactcatgatgggtgtacccgaacattcctgggctcaacaaaaccgattatctttataaccgcggcgcctagcacagcgcctggtgccctaaacgttggctgcgggaacgtccgagacgcgggtgcggagccgggggcggaataactggttgcgcggcgctttgaccgtaggcgctggagcgcgtgcgttgcgtgcgcgcgcggaggcggctgcgtcggggcgcgagaaggtgcagttccccggcgggcgggcgggcgggcgggcgaagctgggctcggggccaagcgaggtctagccggagcgactgtgccccgcctcctgggcggagcgggcggctccccatggtcagagcctcgtgccggctcggcagcgcccggacgccgagcccagcgcgtcggccccccggcgtgcgggcgtctcagagccgcggaggggccgccgggaccgtttcagcgtggcggcgctggtgctggcgttggccctggaggacggccccgagtgatggctggcgcctgcctcccgggtgtctcccgggtacagatggagtcgtcccgcggccgccggcggcaaggtcggcagctgcgaggccaagagagaccccaggacacacacagctgcctcccggtgcgagaagaagaccccggcttgagagtgag

SEQ ID NO: 121 - DNA, 889 bp, Homo sapiens
cggccccatggctccgtgtcgtgtccaagggatgggctggcacctcttggaccaggcttaccaccagggcccttctctgaagccccagtctgaccggcctgctgctgggaatccccctctgcccccacactaacctctgctggggctgagccagggcgcgtcggacagtcagggcgacccagccagggcgaccgttggccccgctcctatggggcagcagggaccgacgtcagcagggtggggcgggcacccgagtggtatgccccgccctgccccgcctgcccgccctggtggccgtctgggggcgacaagtcctgagagaaccagacggaagcgcgctgggactgacacgtggacttgggcggtgctgcccgggtgggtcagcctgggctgggaggcagccccgggacacagctgtgcccacgccgtctgagcaccccaagcccgatgcagccacccccagacgaggcccgcagggacatggccggggacacccagtggtccaggtgtggcgggggtgaggggagggggggtgggagcggtggagatggggccgtggggagggagctgagatactgccacgtgggacgatgctaggtggggagggctgagctgggcgggctcctctggctgtggggccccctgtgttccttgtgggaggtggaaggaagtgagtgccctgtccttcctccctgccatgagattccaggaccggacctggcaagtgccctatcccagccagtgttcctggggctcttccaggcagggctatgttccccaggccaggggcattgtcctggacagtcaggaggcatacccctcgccaggtggaaccaccctgtgtatgcatgaccctgacaagcaggcgccaggacagtcaggaggccag

SEQ ID NO: 122 - DNA, 863 bp, Homo sapiens
gttgttgggtgtgaatggagaactgtgggccctccccgacaccttccagcgggacggcaacgggggcccagggggtgggcgccatcaaccccgtcccaccgccaggacggcgcgggggagggccggcgggggcggggcgtcctgtaaggcgcggcccccacccgcgggcggggcggcattcctgggaggccggcgctctgacgtggacccgggggccgcgggcacggcgggggggcggcggtccgggggcttcttaaaccccccgccccggcccagcccgcacttcccgagcaccgctccgaccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatag

SEQ ID NO: 123 - DNA, 2198 bp, Homo sapiens
aagagaggcacactccctctaccacaccgagggagggggcgttgagctgagaaaggttgagagaatgagggacccaggtaggtggacatcggccaagaaaggaaccacagcgggaggtaagaccgagagtccccagcttgaagcgtcaccactccgggattcccagattccaacgcgagcctggggaaagcccacagtggagagagtccggctggcagggaatggccctacccccggggtgaaatctcggagggtcgtgcagccgagtcgcgcctctgcgctgatgcgtgagagatgccggacgtcgcgtttgcctgtgcgagcctcgcggatgctgtgcagtcttggtcccctctgcgtgtgtctaacgccgaatgctggtgtctcgaggtgtgagcttcggggccggtgtctttaaagaaccaaagattcttaaggagtgatgatctgggtagagcggcccgacgtagccgcgctcccaggtctcggtgcgagtcctgcggacagaccagaggagacctgctggccagatgccccgggcccaaggcggacgccagactgtctctgcgccagccgggctggccttcggaatggatcaggcacccgggaggccggagtggatctcagaccctcaagccgggaacaaacccgtcgatgcccgtgggcctggagtccgcctcctccttcccgccccacccctacccctgcctccgaaaggcttcttcgctggtcagtagctgcgtgcccgtctgcctgaggctgggtcagaattggcgggctggtaacgaccccgtgcacaagcggctcccagtctctccagaaagggccgatgactaaggggtgggggtgggggcggagggctggaaggtgttagggaagaacgttagcggcctatcctgtcttcagcagcgccctctcatcttctagctctgacgccgagcagagcagttggagctcgggactgggaactgctggaattcctatttagacttctagacagtctagaaacaagaacctttctttccctgggcctcagtttccttgtctgtaaaatcaaaaggcgggctctaggtgtaggccttcttttcgcttggtgattctggattcctttccttggatccgtggggagggggtggcagcaacagtccagggcgttggccgtcctgtgcctcaagtacgtagtccccgtgcccgccccctcaacacccccagcagcccgcccccctaagcccgcagagcagggagctgagtgggaggggcagaggcggggccggttcccagtccctgctggcggactagagtggcgcgggctgagcgtaaaacctgggatagccactcccccttttccttatccccgcccccctgccattggctcccgggagaggttgacatcaaagccgcggtcttatataagccagatccgcaggggagtccgcagaagggttaaacaggtctttgggcttcggcgacctcgcccgcggcagaaaccggtaagaagacagtgggctgcgcgtctcattttcagccttgcccggactctcccaaagccggcgcccagtagtggctccagagcccacaggtggcccccggcagtctctggggcgcatggagcggcgttaatagggctggcggcgcaggccagtagccgctccaacatgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctg

SEQ ID NO: 124 - DNA, 1945 bp, Homo sapiens
ctggatgacagagtgagactccgtctcaaaaaaaaagctccatttgggaggccgaggagggtggattacctgaggtcaggagtttgagaccagcctggcccacatagggaaaccccatctctactaaaaatacaaaaattagtcaggtgtggtggctgacacctataatcccagctacttgggaagctgaggcagggagaatcacttgaaccggggaggtggaggttgcagtgagctgagatcatgccactgcactccagcctgggcgacagggtgagattctgtctcaaacaaacaaatttaaaagctccgaatcctccaaaaataccaagattttcctgtcggtaactagagatgggtactgatgattatttttaataggtgattttcaaagatgtgaacgttatccatggagatttaagtctccaaaaggaaaaaaaatgcatacctttatactaaaacttcatcaccagtcaaatttggatcatcactaaattggcttctacacctctctcctaatataaggtacttgtgtaagtttgcagttgtgagacacttatttcctcatttttaatgtcttctcagtagggccactgatatagtcactatttgactgaccagaatggttggcactggtgattggctcataaagtgccctcgatttagggggctcaattatcaaaggtttaaatcctagcccaaaccattgctgtgatgggggttaatcaatgaaccactcagcttcacttgcaaaagcgggatcacaatagccgctttcgtcatgacccagcctaggtgagatttagtacttaagtacactgccaggcacacaaggttaatttaacaatttaacacatttgtttcctcatccatttctccaaaccttccaactaatcctaacgttcgttcggccaaatgggccaggaattcacttaaacaaaaacaaaaaacaaaacaaacaaaaaaacactccctggggcttggggaaggaggcaccgccgcccatgtcgcagtctgggggtggctcagtcctcagcacccagatctacggccataatgctcttcgaggccaaggagcccggatgcggggcgttgccgaaggcgtcttgctcaggctgcgggaaaggagaggggtgggagcggggtgggggcatcgcgacccagggcaaggcggcgagtcgccgtcttcgagtcccacctgtccgaagcggggtgagaaaaggcaaaacatggcaaagccatgcacctcccagggtgggcaactcacggccggtgaacgccggacccttagcagtttccagacctttggaaccggaagcggagcctgagagcgcgcccgagagggcgtgaacgggaccgctttcccggaagtgcttgcggcctctgcccagcgagctgccccggggtctctctggtttcctaatcagggcaacgccgcgggagagaacctttaccttggctgcactaagttctcggtgccactccctggcagggcgggaccttgtttaggccctgtgatcgcgcggttcgtagtagcgcaaggcgcagagtggaccttgacccgcctagggcgggaagagtttggcccgccgggtcccaaagggcagaatggacgggctcctaaatcccagggaatcctctaaattcattgcagaaaacagtcgggatgtgtttattgacagcggaggcgtacggagggtggcagagctgctgctggccaaggcggcggggccagagctgcgcgtggaggggtggaaagcccttcatgagctgaaccccagggcggccgacgaggccgcggtcaactgggtgttcgtgacagacacgctcaacttctccttttggtcggagcaggacgagcacaagtgtgtggtgaggtacagagggaaaacatacagtgggtactggtccctgtgcgccgccgtcaacagagccctcgacgaag

SEQ ID NO: 125 - DNA, 2379 bp, Homo sapiens
aagcttgtggtttacttggacctctgcctcatctttcttcttttgcgcttcagcctgcgcattcgcttcctccactaggctctcatggtgcagaggtttccaagaagatggtgtgaaggccgagatcatttggttatattataaaatagaatgcaaattcacacaagtttttgttttttatttatttatttttttagagatgaggtcttgctatgttgtttagtctggtctcgaactcctggcctcgtgatcctcccaccttgacctcccaaagtgctgggattacaggcctgaggcctgagccactacacccaactgaattcacatttttttttttcttttctgagacggagtctcactctgtcacccagtatggagtgcagtggcgcgactgcggctcactgcaagctccgtctctcgggttcaagtgattctcatgcctcagccccccaagtagctggaattacaggggtgcactaccacacctggctaatttttctgttttagtagagatggggtttcaccatgttgcctggtctcaaactcctgactttaagtgatccacacacctcagcctcccaaagtgctgggattacaggtgtgagcctccacacccggccgaattcacatgaattttaaagtgatgtcttcaaagtggtttcactgtggggatgggcagctttttgttatacatctagaacgttcctcttctgtttctatgaatactcggttggaaagggctgaaaaacggtcttaagagattatctgattcgtttcccagttttattactcacatatcagctgtaatttgagcacgttttctgattgagacaagactcagatggtattaaacattactacaacacatccgggcacggtggctcacgcctgtaatcccagcactttgggaggccgaggcgggcggatcacgaggtcaggagatcgagaccatcctggctaacacggtgaagccctgtctctactaaaaatacaaaaaattaggcgggcatggtggcgggcgcctgtagtcccagctactcgggaggctgaggcaggagaatggcgtgaacccgggaggcggagcttgcagtgagccgagatcgcgccactgcactccagcctgggcgacagagcaagactccatctcaaaaaaaaaaaaaaaaaaaaaaaactacaacactataaattcatatctattataatagtactttgtgcagggccctaccctaagtccttaaccgaacccggaagcgagaagatgacttttgtttgtttttagagatgggcgcctggctctgtcgccagcctggagtgtgggggcgcgatctcgactcacagcagcctccacctcccgagttcaggcgatcttcctgcctcagcccctcgaggagctgggaccaccggcgcgctccatcgcgcccggctaggagctgactttgaatccgggctctgcgcctggccttctgcatctctataagggaagacatctgtgacctcggggcaaaggtcaaattagatcctgggtaggatcctgttcccgctgcccctcgggctggcactgccaggagtactcagagctcaaagctgggatctgcagtcccttacccactcagtgcacgccgcctaaggctttgcgcttcacctttactcacctcgaagccctggacatccgcatctgccctaagacttctcacctcagtagcagaaggaagtcgcgtcagctggccacagcctctctcctaggagaccgtccgggaaaagcgagtcagggtagaccctgaggcccctcagctccggctattttcagatctgtcgctccttcaccctcagcctttcaaacaggccactccaaaaaaaagcccaatcacagccttccttcttctcctggccttccggcactgtccaatcaacgtacgccatctatcggttagtggtgttgcggggccacccttcccgctggtttccctcgtggtgtgtaaaggcagagaggaaaggcgaggggtgttgacgccaggaaggttccatcttggttaagggcaggagtcccttacggacttgtctgaggaaagacaggaaagcgccagcatctccaccttccccggaagcctccctttgccaggcagaaagggtttcccatggggccgcccctggcgccgcgcccggcccacgtacccggggaggccgggccccggaggacgagggaaagcaggccgggcgccgtgagcttcgcggacgtggccgtgtacttctctcccgaggagtgggaatgcctgcggccagcgcagagggccctgtaccgggacgtgatgcgggagaccttcggccacctgggcgcgctgggtgaggccgggccctccggccgggacccccagtccgtcg

SEQ ID NO: 126 - DNA, 933 bp, Homo sapiens
gagacgtactctggctctgtcgcccaggctggagcgcaatggcgccatctcggcgcactgcaacctccacctcccgggttcaagcgattctactgcctcagcctcccgagtagctgggactacaggcgcgcactaccaagcccggctaatttcttttgtatttttagtagagactgggtttcacgatgttggccgggctggtctggaagtcttgacctcaagcgtgcgccctctccgccactgggtaaggcggggggcggaatagggggcttgcaatttcacactagaggcgggcgccgtgggggaaagaagagtcacgtctcccacggttcgtagaggaaggcctgcctgagcctggagcgggggcgggagagccacagtttggcatccccagggcatcccccagcccgcagactaccaggcctccagaggacaggaccccacccccggccacaggccctgcccccagcactccccgcaccccgcctccaagactcctccgcccactccgcacccaacttataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcggctcggccaggggctgcgcgaacacgcggagcgcacccgcagccagctgagcgcgctggagcggcgcc

SEQ ID NO: 127 - DNA, 6096 bp, Homo sapiens
atctgcacctcctcatatagggttgatccaagtttcacagacatcactgagttcttagtggactcagctattggggctgttctcacactttttttttctttgcaagaatcagcaatgggtgcaagtggacctgtgtaggacgtccagtgaaacattgtgttggtgaatcagctagaatccatccaagaactcagccagcctggtgtggggtgagatctgatccttgaatgtccctcagtggcttttagggctggcaggttcagaagggccctctcatcacccccccagggcctcattccttgtttaacactttgctatcacagtcttgaatccttgtaattgaacaatggaccccacattttcactttgcactggtttctgattctgtaaccgatcctgtccccctctcttgtctcattcactctgggaattgtccccacattctgagacctttcagcagtgccccaacgaggttcctgcccttatctgaagctccaccctcacccccatggcggcaccgcaggcagccctgcttttgcgtcccgcgtaggcaggctgtgcaccggagtcacgaccccctgattcagcctaggcagccacagcttgactgctcccgccggacaagccctactgtgctatctgccgctcttcccttcctcttcccagggggtccgcgtcaggggaggcgcagctgtgtgcattccgggagcttcagacccccgtgtccagcagctccttcgtttcctgggtgctggggcggccttcccagcgaagagctcaactcagcgggacgtttggaggctctctgccccaaggcgctggggagtgtgcggcgggacagtcgtgcttgcctttttcactttcagagtgtccacgccccacccgtttggtcactgcaggtcagtccagtccagcccggcccaccccaccggtgcgtgtctgtcgcacgtggcagacgccatactctctgttcttgtttaaagcccaggatctactgggccctggaggcaagaggtgaacgcagcggaatccacgctgagctgcccgggaacggagcttccaaccccagaaggaggactctgtgctcctacaccttaaccctttttagcccgaaacttctccaacttccttggctttgtttagagctcgacagcgccgccccctggcgctcgttgtgaggacagtagaggagagaggcaagggtgtttttaaacagtttgcctctcaccattatgggggcgacccgagggggagacccactcttccgcattcccggtaagtgaaccaccggaagaggtcgaaagtgacggattcccatgtcctcctccagccccccccccaccctgcccatccacaggacggtggctcttcagtgccctttgccgagcaagtggcgtttctatgcacgtgggtatcaattcggactctggacgaaatggaaacctccttagccgacccgggtgggatcagctgggatcctgcgcgctcccctggggggttgccagccactctgttggggtgcaagaagcaccatccttcggaagctgggccgaaactggccaggctgactcgctcccacgcgcccgcccctacccggcgccgcagcaattcacctgccaccgcctctgagccgggtccggacttcggcgccctgacagtgtccccgcgacttccccacccgatgagatggggtctggcgttggccagtgcgtgtccagggactcgcgggtccctggccagccatggggcagagggcgctggtgttaggccagtcttccccaccctgccccgtcaccccagccacacccactgtcctgtgaggccaagcgcgctccgctggtttcctgagccaggcaccttggccgcggacaggatccagctgtctctccttgcgatcctgtcttcggggaagtccacgtcctaggcaggtcctcccaaagtgcccttggtgccgatcacccctcccagcgtcttgcaggtcctgtgcaccacctcccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 128 - DNA, 2500 bp, Homo sapiens
acccacttctgtgtgtggatagtatcctgcaggagagatgttgtctgcagtgtgagctgggcccaccggagtgtgtgaataggatcctgcaggagaaatggaatccggagtgtgagctgcatccgctgtagagggtggataaaatcctgcaggaaagatggcatctggaatgtcagcgggagccaccgacctctgaggatgcaccccgcaggtgtgatgcggggccagttccaaggctgggttaggttttaccctggcttctgtgttgtactctcattctcttcctctttcttctaatacctgctctgggaggcatcaggccatgtccagtgtgcaggccatggagacccacacggcaaggaactggaaccccctgccagcagcctcgggggtccagtccttagatggtgccctgtggtcagcaatgcacctgtgacctccgggctatgtctcgtggtagttgcttttgtgttttaacatagcaacaggaaactagcctattacccaccaatcccattccaggctgctttcaaacgcagctcaggctagaacaccagcacggggacacagctgagacttggggtttgcgacgggaacacgcccatgctgtgcctctgaatctggcaccgtcaccctgtggcctgggttcagcaacttggcctcaccttccttgtctgtgaaattcagactgggtccttgtgagatgattggagagaatgtatgaactatgtgagaacgccacctttgtgcgtatctcacgcagtgtcttccctcctttccaaagtcttctgctgtctctagacacacccgacgtggggggggggggttccctgggtctcctcctaggtctgtcccaggagggcacgcactgaaggccgcgagaatcccgggggctgcattgcgccgcgccaaggactccacacaggacctttcattttcccaactgtgctgagccaggcggccggcagagagcaggtggctgacaggccccggggagccggaccgcctgggtctaatcttcccgcagactcccttgctgtgcgctttggggcttgggcctcagtttcctcaaaaggaatgaggggcttttttggaacgttaaataatttcctacgtggttgcgggtagggagaaggagaaagagaggagcgcgcctgcgcgcctggaatcgtgcccggatcagagcaagcgctctaaaagtgttacaaacattaaggcgccaactaaaaaacccgtagtgagcgcaggcagaaaccacgggtaagagaagtggagaagcttcgcgtaggccccagggtcccgagccccgagtctcgagcgcagaatcaggggtgccaatgctctcctccgcgcccccgagcgctcgccttggccatgcgggccgccccaccgggatgagggcgctcaggccggacgctggggccccgggttctcgccccgccccgccctcggggattcagaggggccgggaggagcctcgcgcatgtgcacagctggcgccccccgccccccgcgcacagctgggacgtgggccgcggccgggcgggcgcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctgtgagtgccgcgggggacgccgggggcgcggggtccggggcttcgtggagatccgggagcgcaggggtgatcggaggtggggggcgcggagggtggagggggcatcgggcgcgcggggggcctggggacttgggacgcagaagggaacctccgaagggggacgtggggggacctgggcgcggggacccgctgggcctttgttcgccctgcgggagacgccgaggggcggaacagagcgctgtgcgcgcggccttcgtagccgcctttgttcggaactcggaatccccgcaggactgggaagttgttggagcctccggggctccccccgctcgcctcccgccgccccctctcatgctccgccggcctcccgcttccccctggttcgcggcccctcctccgctcacctttcccccgctcaggacccctcggtccccctccgctccccgagcgcggcgcagccccctccgtcctcccagccccctccgccccgttcctcgtcctgttcgctcccctcctccgctcctcttcctcctccccttcctcctcctcctccccttcctcctcctcctccccttcctcctcctcctcccttcccctcctcctcccccccttccttctcctcccccagcctccgccctctccccctcccccgccccttggagcgcagtgcccaccccatccccccgcgccgg

SEQ ID NO: 129 - DNA, 2200 bp, Homo sapiens
cctcgccccctccagccggccccccgggcccctcctctcggcgcccggaccttggccctccctctcctttcccacttctctctttgccctaacttcgcccccatcccccgctcatttcctctcgcacccgggctcgccaatccctctttccaagtccctcttccagcccggccttcctctcgggttcgccccccttctccccaatctccgtcctcttccctcccttcgccctcccccccttccttcctcttcccctcacccaaccctggttcccctcgttcctcagtcccgatctctcccttactctgtccccgcccactctgcgccggcctctcagtccgggttgagccccacgtgtggacggccgcgcccccactgacagccgccgcccgccggcccgccccgcgccccgccgggcctctaaaacccccgcgccgcgccctccaccgccgcatcttctccagcgcccagcctcccgccctctctcttgctggccgcacgccccggccccgcgcacctccgcccggctccgcagccgctacccgcgcttcgttgccctgtgggactccgagcgagcccggagggaaccctcctcttcttctgggggcgacttttgtttgcttgcctgtttctttctggtgacttttgcagctttccaatatccgtcttcggagcgcacgggaatccgccgagctctgcgtgcaggcccttttttcttttgaggttcacattttttgaaattttacgccagggcttttgtaatttcctcccccgcccgctgacggtcctggagtcgctcggggctttaggccggttatgcaacgtgtaccgctcggggctgccggctgcacctccgccgcgcctcgccgctcactgcgctagacccggcgccccgcgtctcgcttcgcgggcagtcagggggccggcgctctgtcgaggtctccagctagagcagggagcccgagcccgagggagtccccggagccgacgaagggcttattagaccctgactcttttctgaggcgcgcagattttgtctttgatcactccctctccgcgggtctacggccgcgcgctttcggcgccggcgatggggagaagacggaggctgtgtctccagctctacttcctgtggctgggctgtgtggtgctctgggcgcagggcacggccggccagcctcagcctcctccgcccaagccgccccggccccagccgccgccgcaacaggttcggtccgctacagcaggctctgaaggcgggtttctagcgcccgagtatcgcgaggagggtgccgcagtggccagccgcgtccgccggcgaggacagcaggacgtgctccgagggtaagtgggcaagcggctccgcacctagggctccggcttgggggaggggggaatcctcagtttggcggctttctggcccactccgtcccagaccctttagctggagcctagagctgcagccccctttgccagaatatccaaagacccccaggagcgcgtcccccttttccttcccaaccccgcagctcagcgggcggaaagccctctctccgggggttgggcggcgggtggttagggggtccaggggtgccgatcgcagagcgtgtgcagagctcgcgctgcgggaacaggttctgaatgtccggcggcaggcgggcctgggtccgcctgctgcaggggccagagaagcctgcttgctccccacgtcggggccgccgctcgtgagccttttgtttgaggacgtgtgcagggttcacagctcaccttctcatcgtcaacccgagcgctccaccttgcgacgcgctttccttgacacgtcggggccaaagtaacagttgaccaaggaggaatggatttgggaaggagggcaaggattctttggaacggaatggtccctttgttctctgcatctggaagctagaatagtagcaaattatatgtttccatgcctcttttcgccctttaaaaaggcaggcaagggacgacagatgaaaggcagtgtttagacatttctgaccctcctgcattccagcatctagctcttttgcttccacgtctgcctcccgatctccaataatttgaagtgtaattttgatttgtttgttgtcctgaaatctactcgctcggggcattgcttacgaagaccgtttatatgttgctgcatccctctacctatctgttacgtgaccgcgcttgt

SEQ ID NO: 130 - DNA, 2000 bp, Homo sapiens
ttggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaaggcctccaagatgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccgcgcctcgcttacagctcggccatgctcagctccgccgagagcagccttgacttcagccagtcctcgtccctgctcaacggcggctccggacccggcggcgactacaagctgtcccgctccaacgagaaggagcagctgcaggggctgaacgaccgctttgccggctacatagagaaggtgcactacctggagcagcagaataaggagattgaggcggagatccaggcgctgcggcagaagcaggcctcgcacgcccagctgggcgacgcgtacgaccaggagatccgcgagctgcgcgccaccctggagatggtgaaccacgagaaggctcaggtgcagctggactcggaccacctggaggaagacatccaccggctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccggcgccagctgcagtccaagagcatcgagctagagtcggtgcgcggcaccaaggagtccctggagcggcagctcagcgacatcgaggagcgccacaaccacgacctcagcagctaccaggtaggaaccgcggctgcgcggccagcctgcgccagcgccagcgccgcgcgcccccgacacttgggctcgtgcccaggcgccctctccgccgcgctccctggtggccgctcgctagagcacgcgcgccgcagacctagggtatttgcggatcagcgtcctcgcccatctcatcctccacactccgcccccacccacctgccccagctgctaagggtcttgacctttttcagaaacgtgcatcttttccagttctaattttgcacgcttgcacgtttaaagcaggagggatgaattcggtagtggataa

SEQ ID NO: 131 - DNA, 2300 bp, Homo sapiens
tcagattgtcattgggagggtgaataaatgaatgcttgcattatgagagtttgggggcagaaatatgccacagactcttatctgaagccatcagatttagtggctgcgaacccaccgaagtcagggatttacattttttacagcaacgagagaaaacttcccctttcctctgcagaagtcaggactggatctcaaaaatagaaatgtgtcctcctaaatgtgtgcccatccccgtggttgacaaacaacggatttcccaagatagctgccacacacttggtttctaatctctgtattgcttccccgccagaatgtcgaagtccttcccgaatatgcccagtcatactttctgaacttttgagcaaacaccgtccggcttcttgtgctttcctcaaagaccccaggcaccggcagggaggacacaggccggggcagagcgcccctgcgcgggggattcctgccactccgcgccagcctgcggcgcaaacgctcttctcagccgcagtcccacccgctgctggcaatctgaatgaggagccgcgctatttttacctccccggctgcaatcctttatatttacatgcaggaagcaaatatataagggattaagaaggagatgcgtggccttagtttatccagagcaggaagaggttggaataggagagggtatgtgaagtctggggtggtggaaaaggcaggtggacttcggctggttgttttctcccgatcatccctgtctctggcctggaaacccccgtactctctttcttctggcttatccgtgactgccggctccccctccaccgcccccatcttttgaggtaccacccgtcacctccgatgctgcttgggctgctgcatcactctgctgctttacccccttccccgccccccaacaaagcatgcgcagtgcgttccgggccaggcaacagcagcagcacagcatccagcaacagcatcagcacccgaagccccgctcgggcgcgctctcggggggcggggcgcacgcccgctccgcgcgtccccgcgccgctcgctcccgcgcgtccccgcgccgctcgctcccgcgcgccgcctcagcatcctcaggcccggcggcagcccccgcagtcgctgaagcggccgcgcccgccgggggagggagtagccgctggggaggctccaagttggcggagcggcgaggacccctggactcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcggtgggtatggccccgtgccctttgcgttggctttcccgcggggccctgcagaggaaagcgaagggcgcgcgggtccgtgtgctccgggcttgtccccggctcggcctttccttccctccctgcctgtctttccacccttctcgttcccaaacccccattcatcccagttcacttttggaagtccatttctgttgcattcgcgaaaaacccattccaattcttgttggttccactgggaggtgtttagtggatcctgggtccctcagcgatctctgtgcaacttgcggaggggcaaccagtggatgggaaatacagcgagggagcaagttgctacttgcgtggtggaaccttaatgtgaatgcggggaggatgtagtgataatagtggtaatgggctgtttcctcaaatttcgtatccggcgcattcagtgcggttggaattaaggtgggggaggcacacttcggggaccaaagaattaaggtgctgaagacatacttcatgcacgacctttggttctgatttctcaaagtgcttgtcattataatgaacaattaatataataccatcttctatatattgatgattggaagtcactgaaagcagaaagctggctttgtcaggaaaataaaaagaaattgggaagctgccagcatctgtatccctacatggc

SEQ ID NO: 132 - DNA, 3000 bp, Homo sapiens
tactgccgactttaggtctctctggatctcaggcccccttctctaagatgcatcctagaggaccaaaaatacactttatttgggcttcgcctgcttttgtggaagggtagtttactagaggatataatctcgtgttttaatttgctctctctcctaaaggaaatgtggagaaaaaaaaaaagcagaaattggaaataaccaatatttagtttatttcattcgattcttaggggaactggtgaggagcctaagatgattttcccttcctagagaaagaatccaaagtccagggaaatagcgacaggggagttcaagactgcccctgctagtccttccttggctactctccgctgcgatcgcaggatagctctcattagcaggagaatcgggcaagtgtgtggataagtagagagtgtgttgaacaacttgtaacgttttatgaaatacgcattgtcatggttccctaaaaggctttgcggaagccgtttgtctttactaatcaagtctttacttacacaaaagtagaagtagaagtagttttagaaaacatactaacaatcttctatccccttgaagaccagagtagcagaaaacaggtgatttgcattataaaattgcactcactttttcctcctttcagatttcacattacattagcccatttgtgttacggtgtataaaaaatggaacaggcgcctccactgcattgttctcctttaaaaatagatcacttacaccctaactttgttttccttaaattcgattcttaacaggagagctttctattatttcagatggagtgaggttgcacgactgggatggaagaaaggaatcccttaaatttgggggaatttctgttctctgttccaagaccattttacttggggtgtgggggtgggcgcggcggtcagggcagtggaacgcagtcgcggctgcgccatccctgcacttccaggcgcgcgggagggaccggcggggacgcgagctgcggactctggcgaactcgggggaggcagacagggggaggcggacacccagccggcaggcgtctcagcctccccgcagccggcgggcttttctcctgacagctccaggaaaggcagaccccttccccagccagccaggtaaggtaaagactgctgttgagcttgctgttactgagggcgcacagaccctggggagaccgaagcttgccactgcgggattctgtggggtaacctgggtctacggaagtttcctgaaagaggggagaagggtttgcatttttcctatggaggattcttctctctctagcatttcgtttgatgtattcaactggtagaagtgagatttcaacaggtagcagagagcgctcacgtggaggaggtttggggcgccgcggcgccacccccacccctcctcgggaccgcgcctatttctaaagttacacgtcgacgaactaacctatgctttaaattcctctttccagccccgtgagtccgcggcgacattgggccgtggggtggctgggaacggtcccctcctccggaaaaaccagagaacggcttggagagctgaaacgagcgtccgcgagcaggtccgtgcagaaccgggcttcaggaccgctgagctccgtagggcgtccttgggggacgccaggtcgccggctcctctgccctcgttgagatggacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgccgtgctgtacgtgttgctgcgggcgccccgcatgaagaccgtcaccaacctgttcatcctcaacctggccatcgccgacgagctcttcacgctggtgctgcccatcaacatcgccgacttcctgctgcggcagtggcccttcggggagctcatgtgcaagctcatcgtggctatcgaccagtacaacaccttctccagcctctacttcctcaccgtcatgagcgccgaccgctacctggtggtgttggccactgcggagtcgcgccgggtggccggccgcacctacagcgccgcgcgcgcggtgagcctggccgtgtgggggatcgtcacactcgtcgtgctgcccttcgcagtcttcgcccggctagacgacgagcagggccggcgccagtgcgtgctagtctttccgcagcccgaggccttctggtggcgcgcgagccgcctctacacgctcgtgctgggcttcgccatccccgtgtccaccatctgtgtcctctataccaccctgctgtgccggctgcatgccatgcggctggacagccacgccaaggccctggagcgcgccaagaagcgggtgaccttcctggtggtggcaatcctggcggtgtgcctcctctgctggacgccctaccacctgagcaccgtggtggcgctcaccaccgacctcccgcagacgccgctggtcatcgctatctcctacttcatcaccagcctgagctacgccaacagctgcctcaaccccttcctctacgccttcctggacgccagcttccgcaggaacctccgccagctgataacttgccgcgcggcagcctgactcccccagcgtccggctccgcaactgcccgccactcctggccagcgagggaggagccggcgccagagtgcgggaccagacaggccgcctaggcctcctggggaaaccgactcgcgccccatacccgacctagcagatcggaagcgctgcgactgtgcccgcaggttgaccttgccaagccctccaggtgatgcgcggccatgccgggtgaggagaactgaggctgagatcgccacactgagggctccctaaagccgaggtggaggaagaggagggtagaggaggagggcggtattgctgggaaccgccccctccctgccctgctccctgctgccccacccgagccct

SEQ ID NO: 133 - DNA, 3000 bp, Homo sapiens
gaatacattaaagtaggggcaacccttgagcccagacttctgccatgtgaagaccctttgaaaatcctgacaaacacaggtactgcgtaagtggtcagctaattaaagaggggaggtggagctgtcctttgtgtatccaataagtacccattatctcatttgagcatgaaaagaggccactgttattactttcaagaaggaaagtaagcaggatagctcatatttttagaaccattcctcaccaaatggaataattccggtgaaaagtgggagtgaggaagaaagaaaaaaaaaacttctaatcataatgtttgggaataagaaaggaagaagaaactcacgtcaaagccgactttctcctgcagctgtaaaataaactcttaagacccttcctgctgaaactctggagaggaaaactggagtggcgggtgggctttgcctgcagctcaactctccctcgcggcgcgggcgcggctgggttcagcacctcggaaagcgcccctcgcggcgccccgggattacgcatgctccttggggcccgccgccttggccgtgcaagtgccaccgtaactggtgagagccgctggcaacccacccggagttgacaaccgcggagagacgcagacacccactgacctccaggaagctgagcgtggtggatggaactctacgatctctttctctccaaggacggaaacctcatccaagcagtcccagaggaaacggataaaggtatttgaaagggagcgagcggccccaaatcgcacaattgagcggctgggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgctgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctggggggggccccatttaagaagtaggtgggaggaggatgggcagagcatggaggaggagcctgtggataggccgaggaccttctctggagaggagatgcttcgaaatcaggtggagagaggaaattggcaaagggatagagacgagccccacgggccagacagccaacctccgctccgcaccccacagcctctccttactcttcccacgctgagtagtgtgggggcgcccagaagcgaagacaagcagcaaaaatgtagagaaattggcacggggagcggggcttagccaaatgatgcacagacaattgtgcccgtttattccagcgacttctgcggagagggcagccgtcggcacaaacactcctttgcgt

SEQ ID NO: 134 - DNA, 2200 bp, Homo sapiens
gtcccccgattccctcacccatcatataacgtgtgtatttattatgtttcccgtttcctctgtctccgccagcagaatgtaaactccatgaggtcaggaatctccgagttatgttgcgccagtgtaatccaagagcccggaacagtgcctggcacacagcgggcatatggaagaacaaatgtgtgaaggtgtgaatgaatgaataattgaaagaataaatagtagttctcagcctcacagaacacgggtcacaacctcaaatgacctgctaccctgcccataaataacagagatgcaggagtaagtgctgggctgtgacctgtcaacatgctaagccgctcaaacaaaactgcccaacagcccgctggccgcctatttgcagcactgggccctgagccgcacattcccatttcgttgataaagaaactgaccagatagtttaagtggcctgctgcggaagacagagctggtgctgcaccggtcgctgcttccccagtccttttttggcctcctttctgacgcgacgcagaccccagttctggagagtctgtcactcgctccccgtggtgggagatcagaggcctggtgtccttgggagcggcgagcggtgctcggcgcaggatagaaagggagtgcgcgcccgagtcccccagatccctgggaacccgcgccaccctcccgcccctgcccatccccggccgcgctgtcagtctccattagcgctaacaggctccagacggagcgggccgggcgctgggttaatgcaatcggcgcgttacctggggcgcaggctacattaccagcccggcccccgccaggcacggccagaaccagtcagcccgcgccctgccggccgccccgcgcctccagctcttccccggccccgcccgaacgccacacggcggagcccagccccagcccgcgccctagagcctgccaaggcgccgccggtcgggggccggcagggcgcaaggcaccagggatcccctcgccgccggacacgtgagtgcgccctgagcgcgggacagggctaggtctgcctgggaggcccgggccgagacgcgccagcagagggctagcgagtttgtagtgcagtgacgttaagtgtccgagaaggctcctgtggctgttgaagtgtcgcggacctgagctggggagggggtcggcacgctgccctcagcctcggtgagttcaatcccagccatttggggcaggcgagagtgggtgaacgaggaaaagtgctgcagggtcttcagccgcccccagagggctgtcagaagtctccaactcttgagttccggcgtgccccaacctctgtttccaaatttttccagcggacgcgcgctcttttctgggaaccctgcgtccgctcagcgcgcgctcatcccagtgtctaaggcgctcccgggtggtcttgggagttgcaagtagggaggaacggccgggtaaccacctcttttccctttatccaagcagagcctcggcgtgcccccaggaccggtaaagttcctctcgccagccgcatccatgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgagcgtggggtcgggctgggaatttgaatctgggaggtccactgtctgcagcggtggctgggacaggagctggaatacacacggaagggaggcgaggagacaggggcaaatctggggcgcagaaagaactggacagggctaacgggaaaaaaaaaagattggagtcctctggaaggtcattttcccaggctctttgcagagtacctcgagctcattccagcggaagtgtcaggattgggcaccctggaagcaaaacagcagaagagtgaaatcgagtcatgaccctaaagtcatggtaggggtatggatggaaaggacagaatctggggtgccaggttgggtgggggagcctgaccttttgatggtctgctggaagggaggtggagattccaagagc

(19) United States
(12) Patent Application Publication
(10) Pub. No.: US 2011/0039273 A1
(43) Pub. Date: Feb. 17, 2011

(54) METHOD FOR MEASURING DNA METHYLATION

(75) Inventors: Yoshitaka Tomigahara, Osaka (JP); Hideo Satoh, Osaka (JP); Hirokazu Tarui, Osaka (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: SUMITOMO CHEMICAL COMPANY, LIMITED, Chuo-ku, Tokyo (JP)

(21) Appl. No.: 12/936,296

(22) PCT Filed: Apr. 6, 2009
(86) PCT No.: PCT/JP2009/057411

§ 371 Date: Nov. 2, 2010

(30) Foreign Application Priority Data
Apr. 4, 2008 (JP) 2008-098006

(51) Int. Cl. C12Q 1/68; C07H 21/04; G01N 33/543; G01N 33/53

(52) U.S. Cl. 435/6; 536/23.1; 436/518; 436/94

(57) ABSTRACT

The present invention relates to a method of measuring the content of methylated DNA in a DNA region of interest in a genomic DNA contained in a biological specimen, and so on.

### TECHNICAL FIELD

The present invention relates to a method of measuring the content of methylated DNA in a DNA region of interest in a genomic DNA contained in a biological specimen, and so on.

### BACKGROUND ART

As a method for evaluating the methylation state of DNA in a target DNA region in a genomic DNA contained in a biological specimen, for example, there is known a method of measuring the content of methylated DNA in a target DNA region in a genomic DNA (see, for example, Nucleic Acids Res., 1994, Aug. 11; 22(15): 2990-7, and Proc. Natl. Acad. Sci. U.S.A., 1997, Mar. 18; 94(6): 2284-9 for reference). In such a measuring method, first, it is necessary to extract DNA containing the target DNA region from a DNA sample derived from a genomic DNA, and the extracting operation is complicated.

As a method of measuring the content of methylated DNA in a target region of extracted DNA, for example, (1) a method of amplifying a target region by subjecting the DNA to a chain reaction for DNA synthesis by DNA polymerase after modification of the DNA with a sulfite or the like (Polymerase Chain Reaction; hereinafter also referred to as PCR), and (2) a method of amplifying a target region by subjecting the DNA to PCR after digestion of the DNA using a methylation sensitive restriction enzyme are known. Both of these methods require time and labor for DNA modification for detection of methylation, subsequent purification of the product, preparation of a reaction system for PCR, and checking of DNA amplification.

### DISCLOSURE OF THE INVENTION

It is an object of the present invention to provide a method of measuring the content of methylated DNA in a target DNA region (hereinafter, also referred to as a “target region”) in a genomic DNA contained in a biological specimen in a simple and convenient manner.

That is, the present invention include the following inventions.

### [Invention 1]

A method of quantifying or detecting methylated DNA in a target DNA region possessed by genomic DNA contained in a biological specimen, comprising:

First step of separating double-stranded DNA derived from genomic DNA comprising the target DNA region contained in the biological specimen into single-stranded DNA;

Second step of mixing the single-stranded DNA, a methylated DNA antibody, and a specific oligonucleotide comprising a nucleotide sequence of a part of a complementary sequence to any of the following nucleotide sequences and not inhibiting binding between the methylated target DNA region in the single-stranded DNA and the methylated DNA antibody, to form a complex of the single-stranded DNA, the methylated DNA antibody, and the specific oligonucleotide; and

Third step of detecting or quantifying the methylated DNA in the target DNA region contained in the biological specimen by conducting detection or quantification depending on an identification function that is possessed by the methylated DNA antibody contained in the complex and available for detection:

(1) the nucleotide sequence of SEQ ID NO:1 or a nucleotide sequence having 80% or more sequence identity to the sequence,

(2) the nucleotide sequence of SEQ ID NO:2 or a nucleotide sequence having 80% or more sequence identity to the sequence,

(3) the nucleotide sequence of SEQ ID NO:3 or a nucleotide sequence having 80% or more sequence identity to the sequence,

(4) the nucleotide sequence of SEQ ID NO:4 or a nucleotide sequence having 80% or more sequence identity to the sequence,

(5) the nucleotide sequence of SEQ ID NO:5 or a nucleotide sequence having 80% or more sequence identity to the sequence,

(6) the nucleotide sequence of SEQ ID NO:6 or a nucleotide sequence having 80% or more sequence identity to the sequence,

(7) the nucleotide sequence of SEQ ID NO:7 or a nucleotide sequence having 80% or more sequence identity to the sequence,

(8) the nucleotide sequence of SEQ ID NO:8 or a nucleotide sequence having 80% or more sequence identity to the sequence,

(9) the nucleotide sequence of SEQ ID NO:9 or a nucleotide sequence having 80% or more sequence identity to the sequence,

(10) the nucleotide sequence of SEQ ID NO:10 or a nucleotide sequence having 80% or more sequence identity to the sequence,

(11) the nucleotide sequence of SEQ ID NO:11 or a nucleotide sequence having 80% or more sequence identity to the sequence,

(12) the nucleotide sequence of SEQ ID NO:12 or a nucleotide sequence having 80% or more sequence identity to the sequence,

(13) the complementary sequence to the nucleotide sequence of SEQ ID NO:1 or a nucleotide sequence having 80% or more sequence identity to the complementary sequence,

(11) the complementary sequence to the nucleotide sequence of SEQ ID NO:2 or a nucleotide sequence having 80% or more sequence identity to the complementary sequence,

(15) the complementary sequence to the nucleotide sequence of SEQ ID NO:3 or a nucleotide sequence having 80% or more sequence identity to the complementary sequence,

(16) the complementary sequence to the nucleotide sequence of SEQ ID NO:4 or a nucleotide sequence having 80% or more sequence identity to the complementary sequence,

(17) the complementary sequence to the nucleotide sequence of SEQ ID NO:5 or a nucleotide sequence having 80% or more sequence identity to the complementary sequence,

(18) the complementary sequence to the nucleotide sequence of SEQ ID NO:6 or a nucleotide sequence having 80% or more sequence identity to the complementary sequence,

(19) the complementary sequence to the nucleotide sequence of SEQ ID NO:7 or a nucleotide sequence having 80% or more sequence identity to the complementary sequence,

(20) the complementary sequence to the nucleotide sequence of SEQ ID NO:8 or a nucleotide sequence having 80% or more sequence identity to the complementary sequence,

(21) the complementary sequence to the nucleotide sequence of SEQ ID NO:9 or a nucleotide sequence having 80% or more sequence identity to the complementary sequence,

(22) the complementary sequence to the nucleotide sequence of SEQ ID NO:10 or a nucleotide sequence having 80% or more sequence identity to the complementary sequence,

(23) the complementary sequence to the nucleotide sequence of SEQ ID NO:11 or a nucleotide sequence having 80% or more sequence identity to the complementary sequence, and

(24) the complementary sequence to the nucleotide sequence of SEQ ID NO:12 or a nucleotide sequence having 80% or more sequence identity to the complementary sequence.

### [Invention 2]

The method according to Invention 1, wherein the specific oligonucleotide comprises any of the following nucleotide sequences:

(1) the nucleotide sequence of SEQ ID NO:13,

(2) the nucleotide sequence of SEQ ID NO:14,

(3) the nucleotide sequence of SEQ ID NO:15,

(4) the nucleotide sequence of SEQ ID NO:16,

(5) the nucleotide sequence of SEQ ID NO:17,

(6) the nucleotide sequence of SEQ ID NO:18,

(7) the nucleotide sequence of SEQ ID NO:19,

(8) the nucleotide sequence of SEQ ID NO:20,

(9) the nucleotide sequence of SEQ ID NO:21,

(10) the nucleotide sequence of SEQ ID NO:22,

(11) the nucleotide sequence of SEQ ID NO:23

(12) the nucleotide sequence of SEQ ID NO:24,

(13) the complementary sequence to the nucleotide sequence of SEQ ID NO:13,

(14) the complementary sequence to the nucleotide sequence of SEQ ID NO:14,

(15) the complementary sequence to the nucleotide sequence of SEQ ID NO:15,

(16) the complementary sequence to the nucleotide sequence of SEQ ID NO:16,

(17) the complementary sequence to the nucleotide sequence of SEQ ID NO:17,

(18) the complementary sequence to the nucleotide sequence of SEQ ID NO:18,

(19) the complementary sequence to the nucleotide sequence of SEQ ID NO:19,

(20) the complementary sequence to the nucleotide sequence of SEQ ID NO:20,

(21) the complementary sequence to the nucleotide sequence of SEQ ID NO:21,

(22) the complementary sequence to the nucleotide sequence of SEQ ID NO:22,

(23) the complementary sequence to the nucleotide sequence of SEQ ID NO:23, and

(24) the complementary sequence to the nucleotide sequence of SEQ ID NO:24.

### [Invention 3]

The method according to Invention 1 or 2, wherein a counter oligonucleotide is added in forming the complex in Second step.

### [Invention 4]

The method according to any one of Inventions 1 to 3, wherein formation of the complex in Second step is conducted in a reaction system containing a divalent cation.

### [Invention 5]

The method according to any one of Inventions 1 to 4, wherein the methylated DNA antibody in the complex formed in Second step has been made to bind to a support before Third step.

### [Invention 6]

The method according to any one of Inventions 1 to 5, further comprising, after First step and before Third step, the step of digesting unmethylated single-stranded DNA with a methylation sensitive restriction enzyme capable of digesting single-stranded DNA.

### [Invention 7]

The method according to any one of Inventions 1 to 6, further comprising, after First step and before Third step, the step of digesting unmethylated single-stranded DNA with a methylation-sensitive restriction enzyme in a reaction system containing a specific masking oligonucleotide comprising a recognition sequence for the methylation sensitive restriction enzyme as a part thereof.

### [Invention 8]

The method according to Invention 6, wherein the methylation sensitive restriction enzyme capable of digesting single-stranded DNA is HhaI.

### [Invention 9]

The method according to any one of Inventions 1 to 8, wherein the methylated DNA antibody is a methylcytosine antibody.

### [Invention 10]

The method according to any one of Inventions 1 to 9, wherein the biological specimen is mammalian blood, serum, plasma, bodily fluid, cell lysate or tissue lysate.

### [Invention 11]

The method according to any one of Inventions 1 to 10, wherein the DNA sample derived from genomic DNA contained in the biological specimen is digested in advance with a restriction enzyme recognition cleaving site for which is not present in the target DNA region of the genomic DNA.

### [Invention 12]

The method according to any one of Inventions 1 to 11, wherein the DNA sample derived from genomic DNA contained in the biological specimen has been digested with a methylation sensitive restriction enzyme.

### [Invention 13]

The method according to any one of Inventions 1 to 12, wherein the DNA sample derived from genomic DNA contained in the biological specimen has been digested with a methylation sensitive restriction enzyme in a reaction system containing a specific masking oligonucleotide.

### [Invention 14]

The method according to any one of Inventions 7, 12 and 13, wherein the methylation sensitive restriction enzyme is HpaII or HhaI.

### [Invention 15]

The method according to any one of Inventions 1 to 14, wherein the DNA sample derived from genomic DNA contained in the biological specimen is a DNA sample purified in advance.

### [Invention 16]

The method according to any one of Inventions 1 to 15, wherein the target DNA region possessed by genomic DNA comprises a recognition cleaving site for the methylation sensitive restriction enzyme.

### [Invention 17]

DNA comprising any of the following sequences:

(1) the nucleotide sequence of SEQ ID NO:13,

(2) the nucleotide sequence of SEQ ID NO:14,

(3) the nucleotide sequence of SEQ ID NO:15,

(4) the nucleotide sequence of SEQ ID NO:16,

(5) the nucleotide sequence of SEQ ID NO:17,

(6) the nucleotide sequence of SEQ ID NO:18,

(7) the nucleotide sequence of SEQ ID NO:19,

(8) the nucleotide sequence of SEQ ID NO:20,

(9) the nucleotide sequence of SEQ ID NO:21,

(10) the nucleotide sequence of SEQ ID NO:22,

(11) the nucleotide sequence of SEQ ID NO:23

(12) the nucleotide sequence of SEQ ID NO:24,

(13) the complementary sequence to the nucleotide sequence of SEQ ID NO:13,

(14) the complementary sequence to the nucleotide sequence of SEQ ID NO:14,

(15) the complementary sequence to the nucleotide sequence of SEQ ID NO:15,

(16) the complementary sequence to the nucleotide sequence of SEQ ID NO:16,

(17) the complementary sequence to the nucleotide sequence of SEQ ID NO:17,

(18) the complementary sequence to the nucleotide sequence of SEQ ID NO:18,

(19) the complementary sequence to the nucleotide sequence of SEQ ID NO:19,

(20) the complementary sequence to the nucleotide sequence of SEQ ID NO:20,

(21) the complementary sequence to the nucleotide sequence of SEQ ID NO:21,

(22) the complementary sequence to the nucleotide sequence of SEQ ID NO:22,

(23) the complementary sequence to the nucleotide sequence of SEQ ID NO:23, and

(24) the complementary sequence to the nucleotide sequence of SEQ ID NO:24.

### BRIEF DESCRIPTION OF THE DRAWINGS

FIG. 1 is a drawing showing a result of Example 1. The open box represents a measurement result of an index value having correlation with methylation frequency using genomic DNA of a noncancerous tissue, and the closed box represents a measurement result of an index value having correlation with methylation frequency using genomic DNA extracted from Capan-2 (a cancer cell strain). In the drawing, from the left side, A represents an amount of the methylcytosine antibody binding to the target region detected by using a specific oligonucleotide solution A, B represents an amount of the methylcytosine antibody binding to the target region detected by using a specific oligonucleotide solution B, and C represents an amount of the methylcytosine antibody binding to the target region detected by using a specific oligonucleotide solution C, respectively measured by fluorescence (612 nm).

### MODE FOR CARRYING OUT THE INVENTION

As the “biological specimen”, for example, a cell lysate, a tissue lysate (here the term “tissue” is used in a broad sense including blood and lymph node) or biological samples including bodily fluids such as blood, plasma, serum and lymph, and bodily secretions (urine, milk and so on) and genomic DNA obtained by extracting these biological samples in mammals. Other examples of the biological specimen include samples derived from microorganisms and viruses, and genomic DNA of microorganisms or viruses. When the specimen is human blood, use of the present invention in a health check or a simple clinical examination is expected.

As the DNA sample derived from genomic DNA contained in such a biological specimen, a sample treated with a methylation sensitive restriction enzyme or the like, a sample digested in advance with a restriction enzyme whose recognition cleaving site does not exist in the target DNA region possessed by the genomic DNA, a sample digested with a methylation sensitive restriction enzyme in a reaction system containing a specific masking oligonucleotide, a DNA sample purified in advance, and the like are recited.

For obtaining a genomic DNA from a specimen derived from a mammal, for example, DNA may be extracted using a commercially available DNA extraction kit.

When blood is used as a specimen, plasma or serum is prepared from blood in accordance with a commonly used method, and using the prepared plasma or serum as a specimen, free DNA (including DNA derived from cancer cells such as gastric cancer cells) contained in the specimen is analyzed. This enables analysis of DNA derived from cancer cells such as gastric cancer cells while avoiding DNA derived from hemocytes, and improves the sensitivity of detection of cancer cells such as gastric cancer cells and a tissue containing the same.

Usually, a gene (a genomic DNA) consists of four kinds of bases. In these bases, such a phenomenon is known that only cytosine is methylated, and such methylation modification of DNA is limited to cytosine in a nucleotide sequence represented by 5′-CG-3′ (C represents cytosine, and G represents guanine. Hereinafter, the nucleotide sequence is also referred to as “CpG”). The site to be methylated in cytosine is its position 5. In DNA replication prior to cell division, only cytosine in “CpG” of a template chain is methylated immediately after replication, however, cytosine in “CpG” of a newly-generated strand is immediately methylated by the action of methyltransferase. Accordingly, the methylation state of DNA will be passed to new two sets of DNA even after DNA replication. The term “methylated DNA” in the present invention means DNA occurring by such methylation modification.

It is known that methylation abnormality of DNA occurs in various diseases (for example, cancer), and it is supposed that the level of a particular disease can be measured by detecting the DNA methylation abnormality. For example, when the present invention is practiced for the region where methylation occurs at 100% in a specimen derived from a disease, the amount of methylated DNA quantified or detected will be large, whereas when the present invention is practiced for the region where methylation does not occur at 100% in a specimen derived from a disease, the amount of methylated DNA quantified or detected will be close to approximately zero. For example, when there is a region where a methylation rate is low in a specimen of a healthy subject and high in a specimen of a diseased patient, and if the present invention is practiced for the region, the amount of methylated DNA quantified or detected will be a value near zero in a healthy subject, but will be significantly higher than that value in a diseased patient, so that the level of the disease can be determined based on a difference between these values. The term “level of disease” is similar to the meaning that is generally used in the art, and concretely means, for example, malignancy of a cell when the specimen is a cell, or means, for example, abundance or the like of disease cells in a tissue when the specimen is a tissue. Further, when the specimen is plasma or serum, it means the probability that the individual has the disease. Therefore, by examining the methylation abnormality of the specimen by practice of the present invention, it is possible to diagnose a variety of diseases.

The term “target DNA region” means a DNA region for which presence or absence of methylation of cytosine contained in the region is to be examined, and a DNA region containing at least one cytosine in a nucleotide sequence represented by CpG which is present in a nucleotide sequence of a promoter region, an untranslated region, or a translated region (coding region) of a useful protein gene such as Lysyl oxidase, HRAS-like suppressor, bA305P22.2.1, Gamma filamin, HAND1, Homologue of RIKEN 2210016F16, FLJ32130, PPARG angiopoietin-related protein, Thrombomodulin, p53-responsive gene 2, Fibrillin2, Neurofilament3, disintegrin and metalloproteinase domain 23, G protein-coupled receptor 7, G-protein coupled somatostatin and angiotensin-like peptide receptor, and Solute carrier family 6 neurotransmitter transporter noradrenalin member 2 can be recited. In addition to quantifying or detecting methylated DNA in the target DNA region individually, by using more target DNA regions in one detection system, for example, the quantification accuracy and detection sensitivity will improve accordingly.

To be more specific, when the useful protein gene is a Lysyl oxidase gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Lysyl oxidase gene derived from human, and a promoter region located 5′ upstream of the same can be recited, and more concretely, the nucleotide sequence of SEQ ID NO:119 (corresponding to a nucleotide sequence represented by base No. 16001 to 18661 in the nucleotide sequence described in Genbank Accession No. AF270645) can be recited. In the nucleotide sequence of SEQ ID NO:119, ATG codon encoding methionine at amino terminal of Lysyl oxidase protein derived from human is represented in base No. 2031 to 2033, and a nucleotide sequence of the above exon 1 is represented in base No. 1957 to 2661. Cytosine in the nucleotide sequence represented by CpG which is present in the nucleotide sequence of SEQ ID NO:119, in particular, cytosine in CpG which is present in a region where CpGs are densely present in the nucleotide sequence of SEQ ID NO:119 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1560, 1574, 1600, 1623, 1635, 1644, 1654, 1661, 1682, 1686, 1696, 1717, 1767, 1774, 1783, 1785, 1787, 1795 and so on in the nucleotide sequence of SEQ ID NO:119 can be recited.

Also to be more specific, when the useful protein gene is a HRAS-like suppressor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HRAS-like suppressor gene derived from human, and a promoter region located 5′ upstream of the same can be recited, and more concretely, the nucleotide sequence of SEQ ID NO:120 (corresponding to a nucleotide sequence represented by base No. 172001 to 173953 in the nucleotide sequence described in Genbank Accession No. AC068162) can be recited. In the nucleotide sequence of SEQ ID NO:120, the nucleotide sequence of exon 1 of a HRAS-like suppressor gene derived from human is represented in base No. 1743 to 1953. Cytosine in the nucleotide sequence represented by CpG which is present in the nucleotide sequence of SEQ ID NO:120, in particular, cytosine in CpG which is present in a region where CpGs are densely present in the nucleotide sequence of SEQ ID NO:120 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1316, 1341, 1357, 1359, 1362, 1374, 1390, 1399, 1405, 1409, 1414, 1416, 1422, 1428, 1434, 1449, 1451, 1454, 1463, 1469, 1477, 1479, 1483, 1488, 1492, 1494, 1496, 1498, 1504, 1510, 1513, 1518, 1520 and so on in the nucleotide sequence of SEQ ID NO:120 can be recited.

Also to be more specific, when the useful protein gene is a bA305P22.2.1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a bA305P22.2.1 gene derived from human, and a promoter region located 5′ upstream of the same can be recited, and more concretely, the nucleotide sequence of SEQ ID NO:121 (corresponding to a nucleotide sequence represented by base No. 13001 to 13889 in the nucleotide sequence described in Genbank Accession No. AL121673) can be recited. In the nucleotide sequence of SEQ ID NO:121, ATG codon encoding methionine at amino terminal of bA305P22.2.1 protein derived from human is represented in base No. 849 to 851, and a nucleotide sequence of the above exon 1 is represented in base No. 663 to 889. Cytosine in the nucleotide sequence represented by CpG which is present in the nucleotide sequence of SEQ ID NO:121, in particular, cytosine in CpG which is present in a region where CpGs are densely present in the nucleotide sequence of SEQ ID NO:121 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 335, 337, 351, 363, 373, 405, 424, 427, 446, 465, 472, 486 and so on in the nucleotide sequence of SEQ ID NO:121 can be recited.

Also to be more specific, when the useful protein gene is a Gamma filamin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Gamma filamin gene derived from human, and a promoter region located 5′ upstream of the same can be recited, and more concretely, the nucleotide sequence of SEQ ID NO:122 (corresponding to a complementary sequence to a nucleotide sequence represented by base No. 63528 to 64390 in the nucleotide sequence described in Genbank Accession No. AC074373) can be recited. In the nucleotide sequence of SEQ ID NO:122, ATG codon encoding methionine at amino terminal of Gamma filamin protein derived from human is represented in base No. 572 to 574, and a nucleotide sequence of the above exon 1 is represented in base No. 463 to 863. Cytosine in the nucleotide sequence represented by CpG which is present in the nucleotide sequence of SEQ ID NO:122, in particular, cytosine in CpG which is present in a region where CpGs are densely present in the nucleotide sequence of SEQ ID NO:122 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 333, 337, 350, 353, 360, 363, 370, 379, 382, 384, 409, 414, 419, 426, 432, 434, 445, 449, 459, 472, 474, 486, 490, 503, 505 and so on in the nucleotide sequence of SEQ ID NO:122 can be recited.

Also to be more specific, when the useful protein gene is a HAND1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HAND1 gene derived from human, and a promoter region located 5′ upstream of the same can be recited, and more concretely, the nucleotide sequence of SEQ ID NO:123 (corresponding to a complementary sequence to a nucleotide sequence represented by base No. 24303 to 26500 in the nucleotide sequence described in Genbank Accession No. ACO26688) can be recited. In the nucleotide sequence of SEQ ID NO:123, ATG codon encoding methionine at amino terminal of HAND1 protein derived from human is represented in base No. 1656 to 1658, and a nucleotide sequence of the above exon 1 is represented in base No. 1400 to 2198. Cytosine in the nucleotide sequence represented by CpG which is present in the nucleotide sequence of SEQ ID NO:123, in particular, cytosine in CpG which is present in a region where CpGs are densely present in the nucleotide sequence of SEQ ID NO:123 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1153, 1160, 1178, 1187, 1193, 1218, 1232, 1266, 1272, 1292, 1305, 1307, 1316, 1356, 1377, 1399, 1401, 1422, 1434 and so on in the nucleotide sequence of SEQ ID NO:123 can be recited.

Also to be more specific, when the useful protein gene is a Homologue of RIKEN 2210016F16 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Homologue of RIKEN 2210016F16 gene derived from human, and a promoter region located 5′ upstream of the same can be recited, and more concretely, the nucleotide sequence of SEQ ID NO:124 (corresponding to a complementary nucleotide sequence to a nucleotide sequence represented by base No. 157056 to 159000 in the nucleotide sequence described in Genbank Accession No. AL354733) can be recited. In the nucleotide sequence of SEQ ID NO:124, a nucleotide sequence of exon 1 of a Homologue of a RIKEN 2210016F16 gene derived from human is represented in base No. 1392 to 1945. Cytosine in the nucleotide sequence represented by CpG which is present in the nucleotide sequence of SEQ ID NO:124, in particular, cytosine in CpG which is present in a region where CpGs are densely present in the nucleotide sequence of SEQ ID NO:124 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1172, 1175, 1180, 1183, 1189, 1204, 1209, 1267, 1271, 1278, 1281, 1313, 1319, 1332, 1334, 1338, 1346, 1352, 1358, 1366, 1378, 1392, 1402, 1433, 1436, 1438 and so on in the nucleotide sequence of SEQ ID NO:124 can be recited.

Also to be more specific, when the useful protein gene is a FLJ32130 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a FLJ32130 gene derived from human, and a promoter region located 5′ upstream of the same can be recited, and more concretely, the nucleotide sequence of SEQ ID NO:125 (corresponding to a complementary nucleotide sequence to a nucleotide sequence represented by base No. 1 to 2379 in the nucleotide sequence described in Genbank Accession No. AC002310) can be recited. In the nucleotide sequence of SEQ ID NO:125, ATG codon encoding methionine at amino terminal of FLJ32130 protein derived from human is represented in base No. 2136 to 2138, and a nucleotide sequence assumed to be the above exon 1 is represented in base No. 2136 to 2379. Cytosine in the nucleotide sequence represented by CpG which is present in the nucleotide sequence of SEQ ID NO:125, in particular, cytosine in CpG which is present in a region where CpGs are densely present in the nucleotide sequence of SEQ ID NO:125 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1714, 1716, 1749, 1753, 1762, 1795, 1814, 1894, 1911, 1915, 1925, 1940, 1955, 1968 and so on in the nucleotide sequence of SEQ ID NO:125 can be recited.

Also to be more specific, when the useful protein gene is a PPARG angiopoietin-related protein gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a PPARG angiopoietin-related protein gene derived from human, and a promoter region located 5′ upstream of the same can be recited, and more concretely, the nucleotide sequence of SEQ ID NO:126 can be recited. In the nucleotide sequence of SEQ ID NO:126, ATG codon encoding methionine at amino terminal of PPARG angiopoietin-related protein derived from human is represented in base No. 717 to 719, and a nucleotide sequence of the 5′ side part of the above exon 1 is represented in base No. 1957 to 2661. Cytosine in the nucleotide sequence represented by CpG which is present in the nucleotide sequence of SEQ ID NO:126, in particular, cytosine in CpG which is present in a region where CpGs are densely present in the nucleotide sequence of SEQ ID NO:126 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 35, 43, 51, 54, 75, 85, 107, 127, 129, 143, 184, 194, 223, 227, 236, 251, 258 and so on in the nucleotide sequence of SEQ ID NO:126 can be recited.

Also to be more specific, when the useful protein gene is a Thrombomodulin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Thrombomodulin gene derived from human, and a promoter region located 5′ upstream of the same can be recited, and more concretely, the nucleotide sequence of SEQ ID NO:127 (corresponding to a nucleotide sequence represented by base No. 1 to 6096 in the nucleotide sequence described in Genbank Accession No. AF495471) can be recited. In the nucleotide sequence of SEQ ID NO:127, ATG codon encoding methionine at amino terminal of Thrombomodulin protein derived from human is represented in base No. 2590 to 2592, and a nucleotide sequence of the above exon 1 is represented in base No. 2048 to 6096. Cytosine in the nucleotide sequence represented by CpG which is present in the nucleotide sequence of SEQ ID NO:127, in particular, cytosine in CpG which is present in a region where CpGs are densely present in the nucleotide sequence of SEQ ID NO:127 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1551, 1571, 1579, 1581, 1585, 1595, 1598, 1601, 1621, 1632, 1638, 1645, 1648, 1665, 1667, 1680, 1698, 1710, 1724, 1726, 1756 and so on in the nucleotide sequence of SEQ ID NO:127 can be recited.

Also to be more specific, when the useful protein gene is a p53-responsive gene 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a p53-responsive gene 2 gene derived from human, and a promoter region located 5′ upstream of the same can be recited, and more concretely, the nucleotide sequence of SEQ ID NO:128 (corresponding to a complementary sequence to a nucleotide sequence represented by base No. 113501 to 116000 in the nucleotide sequence described in Genbank Accession No. AC009471) can be recited. In the nucleotide sequence of SEQ ID NO:128, a nucleotide sequence of exon 1 of a p53-responsive gene 2 gene derived from human is represented in base No. 1558 to 1808. Cytosine in the nucleotide sequence represented by CpG which is present in the nucleotide sequence of SEQ ID NO:128 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1282, 1284, 1301, 1308, 1315, 1319, 1349, 1351, 1357, 1361, 1365, 1378, 1383 and so on in the nucleotide sequence of SEQ ID NO:128 can be recited.

Also to be more specific, when the useful protein gene is a Fibrillin2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Fibrillin2 gene derived from human, and a promoter region located 5′ upstream of the same can be recited, and more concretely, the nucleotide sequence of SEQ ID NO:129 (corresponding to a complementary sequence to a nucleotide sequence represented by base No. 118801 to 121000 in the nucleotide sequence described in Genbank Accession No. AC113387) can be recited. In the nucleotide sequence of SEQ ID NO:129, a nucleotide sequence of exon 1 of a Fibrillin2 gene derived from human is represented in base No. 1091 to 1345. Cytosine in the nucleotide sequence represented by CpG which is present in the nucleotide sequence of SEQ ID NO:129 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 679, 687, 690, 699, 746, 773, 777, 783, 795, 799, 812, 823, 830, 834, 843 and so on in the nucleotide sequence of SEQ ID NO:129 can be recited.

Also to be more specific, when the useful protein gene is a Neurofilament3 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Neurofilament3 gene derived from human, and a promoter region located 5′ upstream of the same can be recited, and more concretely, the nucleotide sequence of SEQ ID NO:130 (corresponding to a complementary sequence to a nucleotide sequence represented by base No. 28001 to 30000 in the nucleotide sequence described in Genbank Accession No. AF106564) can be recited. In the nucleotide sequence of SEQ ID NO:130, a nucleotide sequence of exon 1 of a Neurofilament3 gene derived from human is represented in base No. 614 to 1694. Cytosine in the nucleotide sequence represented by CpG which is present in the nucleotide sequence of SEQ ID NO:130 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 428, 432, 443, 451, 471, 475, 482, 491, 499, 503, 506, 514, 519, 532, 541, 544, 546, 563, 566, 572, 580 and so on in the nucleotide sequence of SEQ ID NO:130 can be recited.

Also to be more specific, when the useful protein gene is a disintegrin and metalloproteinase domain 23 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a disintegrin and metalloproteinase domain 23 gene derived from human, and a promoter region located 5′ upstream of the same can be recited, and more concretely, the nucleotide sequence of SEQ ID NO:131 (corresponding to a nucleotide sequence represented by base No. 21001 to 23300 in the nucleotide sequence described in Genbank Accession No. AC009225) can be recited. In the nucleotide sequence of SEQ ID NO:131, a nucleotide sequence of exon 1 of a disintegrin and metalloproteinase domain 23 gene derived from human is represented in base No. 1194 to 1630. Cytosine in the nucleotide sequence represented by CpG which is present in the nucleotide sequence of SEQ ID NO:131 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 998, 1003, 1007, 1011, 1016, 1018, 1020, 1026, 1028, 1031, 1035, 1041, 1043, 1045, 1051, 1053, 1056, 1060, 1066, 1068, 1070, 1073, 1093, 1096, 1106, 1112, 1120, 1124, 1126 and so on in the nucleotide sequence of SEQ ID NO:131 can be recited.

Also to be more specific, when the useful protein gene is a G protein-coupled receptor 7 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a G protein-coupled receptor 7 gene derived from human, and a promoter region located 5′ upstream of the same can be recited, and more concretely, the nucleotide sequence of SEQ ID NO:132 (corresponding to a nucleotide sequence represented by base No. 75001 to 78000 in the nucleotide sequence described in Genbank Accession No. AC009800) can be recited. In the nucleotide sequence of SEQ ID NO:132, a nucleotide sequence of exon 1 of a G protein-coupled receptor 7 gene derived from human is represented in base No. 1666 to 2652. Cytosine in the nucleotide sequence represented by CpG which is present in the nucleotide sequence of SEQ ID NO:132 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1480, 1482, 1485, 1496, 1513, 1526, 1542, 1560, 1564, 1568, 1570, 1580, 1590, 1603, 1613, 1620 and so on in the nucleotide sequence of SEQ ID NO:132 can be recited.

Also to be more specific, when the useful protein gene is a G-protein coupled somatostatin and angiotensin-like peptide receptor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a G-protein coupled somatostatin and angiotensin-like peptide receptor gene derived from human, and a promoter region located 5′ upstream of the same can be recited, and more concretely, the nucleotide sequence of SEQ ID NO:133 (corresponding to a complementary sequence to a nucleotide sequence represented by base No. 57001 to 60000 in the nucleotide sequence described in Genbank Accession No. AC008971) can be recited. In the nucleotide sequence of SEQ ID NO:133, a nucleotide sequence of exon 1 of a G-protein coupled somatostatin and angiotensin-like peptide receptor gene derived from human is represented in base No. 776 to 2632. Cytosine in the nucleotide sequence represented by CpG which is present in the nucleotide sequence of SEQ ID NO:133 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 470, 472, 490, 497, 504, 506, 509, 514, 522, 540, 543, 552, 566, 582, 597, 610, 612 and so on in the nucleotide sequence of SEQ ID NO:133 can be recited.

Also to be more specific, when the useful protein gene is a Solute carrier family 6 neurotransmitter transporter noradrenalin member 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Solute carrier family 6 neurotransmitter transporter noradrenalin member 2 gene derived from human, and a promoter region located 5′ upstream of the same can be recited, and more concretely, the nucleotide sequence of SEQ ID NO:134 (corresponding to a complementary sequence to a nucleotide sequence represented by base No. 78801 to 81000 in the nucleotide sequence described in Genbank Accession No. AC026802) can be recited. In the nucleotide sequence of SEQ ID NO:134, a nucleotide sequence of exon 1 of a Solute carrier family 6 neurotransmitter transporter noradrenalin member 2 gene derived from human is represented in base No. 1479 to 1804. Cytosine in the nucleotide sequence represented by CpG which is present in the nucleotide sequence of SEQ ID NO:134 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1002, 1010, 1019, 1021, 1051, 1056, 1061, 1063, 1080, 1099, 1110, 1139, 1141, 1164, 1169, 1184 and so on in the nucleotide sequence of SEQ ID NO:134 can be recited.

As concrete examples of the “target DNA region” in genomic DNA, the following nucleotide sequences of SEQ ID NOs:1 to 12, and complementary sequences thereof can be recited, and also nucleotide sequences having sequence identity of 80% or more, 90% or more, 95% or more or 98% or more to these nucleotide sequences can be recited.

SEQ ID NO:1 corresponds to the nucleotide sequence represented by base No.21965410 to 21964273 in the nucleotide sequence described in Genbank Accession No.NT_{—}008413.17.

SEQ ID NO:2 corresponds to the nucleotide sequence represented by base No.29857545 to 29858420 in the nucleotide sequence described in Genbank Accession No.NT_{—}024524.13.

SEQ ID NO:3 corresponds to the nucleotide sequence represented by base No.7188979 to 7189574 in the nucleotide sequence described in Genbank Accession No.NT_{—}010718.15.

SEQ ID NO:4 corresponds to the nucleotide sequence represented by base No.7534656 to 7535486 in the nucleotide sequence described in Genbank Accession No.NW_{—}926584.1.

SEQ ID NO:5 corresponds to the nucleotide sequence represented by base No.21984168 to 21984790 in the nucleotide sequence described in Genbank Accession No.NT_{—}008413.17.

SEQ ID NO:6 corresponds to the nucleotide sequence represented by base No.21818162 to 21818836 in the nucleotide sequence described in Genbank Accession No.NW_{—}924062.1.

SEQ ID NO:7 corresponds to the nucleotide sequence represented by base No.21998877 to 21999061 in the nucleotide sequence described in Genbank Accession No.NT_{—}008413.17.

SEQ ID NO:8 corresponds to the nucleotide sequence represented by base No.19277002 to 19277451 in the nucleotide sequence described in Genbank Accession No.NT_{—}023935.17.

SEQ ID NO:9 corresponds to the nucleotide sequence represented by base No.19064265 to 19065579 in the nucleotide sequence described in Genbank Accession No.NW_{—}924484.1.

SEQ ID NO:10 corresponds to the nucleotide sequence represented by base No.50318280 to 50318573 in the nucleotide sequence described in Genbank Accession No.NT_{—}022517.17.

SEQ ID NO:11 corresponds to the nucleotide sequence represented by base No.61176814 to 61177405 in the nucleotide sequence described in Genbank Accession No.NT_{—}022517.17.

SEQ ID NO:12 corresponds to the nucleotide sequence represented by base No.14488398 to 14489126 in the nucleotide sequence described in Genbank Accession No.NT_{—}034772.5.

As the “target DNA region” (hereinafter, also referred to as a “target region”), a DNA region containing at least one cytosine in a nucleotide sequence represented by CpG which is present in a nucleotide sequence of a promoter region, an untranslated region, or a translated region (coding region) of a gene such as MLH1, RUNX3, CDH1, TIMP3, CSPG, RARβ, 14-3-3σ, CALCA, HIC1, ESR1, PTEN, SOCS1, BLT1, ESR2, MTMG, TWIST, INK4, CDKN2, GSTP, DCR2, TP73, PGR, HIC2, MTHFR, TFF1, MLLT7, SLC5A8, THBS1, SOCS2, ACTB, CDH13, FGF18, GSTM3, HSD17B4, HSPA2, PPP1R13B, PTGS2, SYK, TERT, TITF1, BRACA1, AATF, ABCB1, ABCC1, ABI1, ABL1, AF1Q, AF3P21, AF4, AF9, AFF3, AKAP12, AKAP9, ALEX3, ALK, ALOX15, APAF1, APC, ARHA, ARHGAP26, ARHGEF12, ARNT, ATBF1, ATF1, ATM, AXIN2, BCAS3, BCAS4, BCL1, BCL10, BCL11A, BCL11B, BCL2, BCL5, BCL7A, BCR, BIRC3, BMPR1A, BRCA2, BRD4, BRIP1, BTG1, BUB1B, CAGE-1, CARS, CASC5, CCDC6, CCND2, CCND3, CDH11, CDKN1B, CDKN2A, CDX2, CEP110, CKN1, CLP1, CLTC, CLTCL1, CNC, COL1A1, COX6C, CREBBP, CXXC6, DAB2IP, DDIT3, DDX43, DIRC1, DIRC2, DKK1, E2F3, EEN, EGFR, ELL, EPS15, ERBB2, ERC1, ERCC1, ERCC4, ERG, ETV1, ETV6, EVI1, EWSR1, EXT1, EXT2, FANCA, FANCD2, FANCF, FAS, FBP17, FCRL4, FEV, FGFR1, FHIT, FLI1, FOXO3A, FUS, FVT1, GAS7, GLI1, GNAS, GOLGA5, GOPC, GRB2, HCMOGT-1, HIST1H4I, HLF, HMGA2, HOXA13, HOXC11, HOXC13, HOXD13, HSPBAP1, HSPCB, HSPD1, HSPH1, IKZF2, INTS6, IRF4, JAG1, JAG2, JAK2, JARID1A, JAZF1, JMJD2C, JUN, KIT, KITLG, KLF5, KLF6, LASP1, LDB1, LHFP, LMO1, LMO2, LPHN2, LPP, LYL1, MADH4, MAF, MAFB, MDM2, MDS2, MET, MKL1, MLF1, MLH1, MLL, MLLT10, MMP2, MN1, MRE11B, MSF, MSH2, MSH6, MSI2, MUC1, MUTYH, MXI1, MYC, MYH9, MYST3, NF1, NFKB1, NIN, NKX2-5, NONO, NOTCH1, N-RAS, NTRK3, NUMA1, NUP214, NUP98, OLIG2, P53, PALB2, PAX2, PAX5, PAX7, PAX9, PBX1, PCM1, PCSK7, PDGFB, PDGFRA, PDGFRB, PHOX2B, PICALM, PLAG1, PLCB1, PLK, PML, PMS1, POLH, POU5F1P1, POU6F2, PPP1R13L, PRDM16, PRRX1, PSIP1, PTCH, RABEP1, RAD51L1, RAD53, RANBP17, RAP1GDS1, RAP2B, RARA, RASSF1, RB1, RBL2, RBM15, RBM5, RCHY1, RECQL, RECQL5, RET, RUNX, RUNX1T1, SBDS, SDHC, SDHD, SET, SHH, SIL, SIX1, SNAI2, SPI1, SPINK7, STARD3, STAT3, STK11, STK4, SUFU, SYK, SYNPO2, TBX2, TCF3, THBS2, THRAP3, TMPRSS2, TNF, TOP1, TPM4, TPR, TRIM24, TRIM33, TRIP11, TSC1, TSC2, TSHR, TTL, VAV1, VHL, WFDC1, WT1, WWOX, XPC, ZBTB16, ZNF146 and ZNF217 can be recited. In addition to quantifying or detecting methylated DNA in the target DNA region individually, by using more target DNA regions in one detection system, for example, the quantification accuracy and detection sensitivity will improve accordingly.

The “target DNA region” may be a repetitive sequence interspersed in a genome. In particular, a methylation degree of a repetitive sequence that is apparently methylated by affection can be utilized as a surrogate marker of the disease. For example, in cancer, methylation of SINE and LINE is known, and concretely, subfamilies such as AluJb, AluJo, AluSc, AluSg, AluSg/x, AluSp, AluSq, AluSq/x, AluSx, AluY, FRAM, FLAM_A, MIR, MIRb, MIR3, L1Med, L1M4, L1M5, L1MA3, L1MA7, L1MA9, L1MC, L1MC2, L1MC4, L1MC4a, L1MC5, L1MDa, L1MD1, L1MD2, L1MEe, L1ME3A, L1PA15, L1PREC2, L2, L3, and HAL1 are known.

Repetitive sequences in a genome are generally classified into simple repetitive sequences (called a tandem repetitive sequence, or a tandem repeat) and interspersed repetitive sequences.

Simple repetitive sequences are characterized in that the same sequences neighbor in the same orientation, and for example, broadly classified into the following five kinds: (1) repetitive sequences in which a relatively short sequence such as CCA is repeated, (2) repetitive sequences derived from a transcription factor, (3) a series of repetitive sequences such as centromere, telomere, kinetochore, and ribosome group genes, (4) inert processed pseudogenes reversely transferred from RNA or protein, and (5) gene sequences amplified by gene duplication.

First, as a repeat composed of a relatively short nucleotide sequence, sequences such as (A)n, (T)n, (GA)n, (CA)n, (TAA)n, (GGA)n, (CAGC)n, (CATA)n, (GAAA)n, (TATG)n, (TTTG)n, (TTTA)n, (TTTC)n, (TAAA)n, (TTCA)n, (TATAA)n, (TCTCC)n, (TTTCC)n, (TTTAA)n, (TTTTC)n, (TTTTA)n, (TTTTG)n, (CAAAA)n, (CACCC)n, (TATATG)n, (CATATA)n, (TCTCTG)n, (AGGGGG)n, (CCCCCA)n, and (TGGGGG)n (n means the number of repetition) are known. Next, as the sequence derived from a transcription factor, MER1-Charlie and Zaphod of hAT group, and MER2-Tigger, Tc-1 and Mariner of Tc-1 group are appropriate. As other repetitive sequences derived from a transcription factor on a genome, Tigger1, Tigger2a, Tigger5, Charlie4a, Charlie7 and the like are known. Since these repetitive sequences formed of a short nucleotide sequence, or repetitive sequences derived from a transcription factor are generally short and simple nucleotide sequences, setting of a specific oligonucleotide of the present method is difficult, however, if a practicable specific oligonucleotide can be set, these are not necessarily excluded as an object of the present method. Here, satellite DNA, minisatellite, microsatellite and the like are repetitive sequences that are classified into simple repetitive sequences.

As to a sequence that exists in multicopy in genes, if a specific oligonucleotide of the present method can be set for genes existing in multicopy in a genome as a result of gene duplication, as well as ALR6 as a sequence existing in centromere, U2 and U6 as snRNAs, and other genes that are known to exist in multicopy in a genome such as tRNA and rRNA, the detection sensitivity can be improved compared to detecting one gene in one genome.

It is also known that a retrovirus, a retrotransposon having LTR (Long terminal repeat) in its terminal, an endogenous sequence such as MaLRs (Mammalian apparent LTR-Retrotransposons) considered to be derived from viruses, and LTR derived from a retrovirus exist in multicopy in one genome.

For example, as the LTR derived from a retrovirus, concretely, subfamilies such as LTR1, LTR1B, LTR5, LTR7, LTR8, LTR16A1, LTR16A1, LTR16C, LTR26, LTR26E, MER48, and MLT2CB are known. The LTRs derived from a retrotransposon are classified into classes of ERV, ERVK and ERVL, and concrete examples include subfamilies such as LTR8A, LTR28, MER21B, MER83, MER31B, MER49, MER66B, HERVH, ERVL, LTR16A1, LTR33A, LTR50, LTR52, MLT2A1, MLT2E, MER11C, and MER11C. Further, MaLRs indicate DNA factors including LTRs in both ends likewise a typical retrotransposon, wherein an internal sequence sandwiched between LTRs is not derived from a retrovirus. For example, subfamilies such as MLT1A1, MLT1A2, MLT1B, MLT1C, MLT1D, MLT1F, MLT1G, MLT1H, MLT1J, MLT1K, MLT1I, MLT2CB, MSTA, MSTA-int, MSTB, THE1A, THE1B, THE1B-internal, and THE1 can be recited.

The interspersed repetitive sequences are characterized by being interspersed without neighboring each other, and are considered to be derived from a retrotransposon. Further, the interspersed repetitive sequences are classified into SINE (Short Interspersed Repetitive Element: short chain interspersed repetitive sequences) and LINE (Long Interspersed Elements: long-chain interspersed repetitive sequences) according to the length. Most of SINES are sequences belonging to the Alu family. A common feature of the Alu family is that it has a sequence of 3′-side or a sequence of 5′-side of 7SL RNA, and that it has an AT-Rich region sandwiched between a Left-monomer and a Right-monomer. As subfamilies of the Alu family, Alu, AluJb, AluJo, AluSc, AluSg, AluSp, AluSq, AluSx, AluY, and FAM (Fossil Alu Monomer), FLAM (Free Left Alu Monomer) having a sequence of FAM, and FRAM (Free Right Alu Monomer) can be recited. As SINEs other than the Alu family, MIR, and Ther/MIR3 are known, and MIR and MIR3 are known as respective subfamilies. As subfamilies of the Alu family including other biological species than human being, B1, B2, B4, PB1, PB1D and so on are known. As LINEs, subfamilies of LINE1 to Line23 are reported, and it is known that subfamilies such as LINE-1, LINE2, and LINE3 broadly exist in a genome. As for LINE-1, for example, L1M1, L1M2, L1M3, L1M3d, L1M4, L1M4c, L1MA2, L1MA7, L1MA8, L1MA9, L1MB1, L1MB1, L1MB3, L1MB4, L1MB5, L1MB6, L1MB7, L1MCa, L1MCb, L1MC2, L1MC3, L1MC4, L1MC4a, L1MC5, L1MDa, L1ME, L1MEc, L1MEd, L1MEg, L1ME1, L1ME2, L1ME3, L1ME3A, L1ME3B, L1ME4a, L1PB3, L1P4, L1PA2, L1PA3, L1PA4, L1PA5, L1PA6, L1PA7, L1PA10, L1PA12, L1PA13, L1PA14, L1PA16, L1PB1, L1PB3, L1PB4, L1PREC2, and HAL1 are known, and as LINE-2, subfamilies such as L2 and L2c are known.

Among these repetitive sequences interspersed in a genome, the one that will be methylated by affection can be utilized as a highly sensitive detection system as a surrogate marker of the disease by the present method.

The term quantification in “quantify or detect DNA” is estimation of methylation frequency or an index value correlated therewith in the target DNA region in the specimen, from the quantified methylated DNA antibody, and means methylation frequency or an index value correlated therewith of DNA of the target region contained in 1 mL of serum when the specimen is 1 mL of serum.

The term “detect” in “quantify or detect DNA” means that whether methylated DNA exists in the target DNA region can be distinguished from whether the methylated DNA antibody is detected or not, and represents that methylated DNA exists in the target DNA region in the specimen when the methylated DNA antibody is detected, and represents that the abundance of methylated DNA in the target DNA region in the specimen is less than a detection limit when the methylated DNA antibody or the specific oligonucleotide is not detected.

In First step, double-stranded DNA derived from genomic DNA that comprises the target DNA region and is contained in the biological specimen is temporarily separated into a single-stranded state. Concretely, for example, by adding an annealing buffer to the double-stranded DNA, a mixture is obtained. Next, the obtained mixture is boiled at 95° C. for about 30 seconds, and then rapidly cooled on ice chilled water for several minutes. For example, free DNA contained in blood or the like can be single-stranded DNA. Therefore, when genomic DNA contained in the biological specimen is single-stranded DNA, this operation is not required.

The term “methylated DNA antibody” in Second step means an antibody that binds with a methylated base in DNA as an antigen. More desirably, it is an antibody that has a property of binding by recognition of cytosine whose 5-position is methylated in single-stranded DNA, and more concretely it may be the methylcytosine antibody. Also a commercially available methylated DNA antibody capable of specifically recognizing DNA in a methylation state described in the present invention and specifically binding thereto is applicable. The methylated DNA antibody may be produced by a usual method from a methylated base, methylated DNA or the like as an antigen. Concretely, it may be produced by selection according to the specific binding to methylcytosine in DNA as an index, from antibodies generated by using 5-methylcytidine, 5-methylcytosine, or DNA and the like containing 5-methylcytosine as an antigen. As antibodies that can be obtained by immunizing an animal with an antigen, a method using an antibody of IgG fraction (polyclonal antibody) following immunization with a purified antigen and a method using an antibody producing a single clone (monoclonal antibody) are known. In the present invention, since an antibody capable of specifically recognizing methylated DNA or methylcytosine is desired, use of a monoclonal antibody is desired.

As a method of preparing a monoclonal antibody, a procedure based on a cell fusion method can be recited. For example, in the cell fusion method, a hybridoma is prepared by allowing cell fusion between a spleen cell (B cell) derived from an immunized mouse and a myeloma cell, and an antibody produced by the hybridoma is selected for preparation of a methyl cytosine antibody (monoclonal antibody). When a monoclonal antibody is prepared by a cell fusion method, it is not necessary to purify an antigen, and for example, a mixture of 5-methyl cytidine, 5-methyl cytosine or DNA or the like containing 5-methyl cytosine may be administered as an antigen to an animal used for immunization. As an administration method, 5-methyl cytidine, 5-methyl cytosine or DNA or the like containing 5-methyl cytosine is directly administered to a mouse for production of an antibody. When an antibody is difficult to be produced, an antigen bound to a support may be used for immunization. Also, by thoroughly mixing an adjuvant solution (prepared, for example, by mixing liquid paraffin and Aracel A, and mixing killed tubercle bacilli as an adjuvant) and an antigen, and immunizing via liposome incorporating the same, immunity of an antigen can be improved. Also a method involving adding equivalent amounts of a solution containing an antigen and an adjuvant solution, fully emulsifying them, and subcutaneously or intraperitoneally injecting the resultant mixture to a mouse, and a method of adding killed Bordetella pertussis as an adjuvant after mixing well with alum water are known. A mouse may be boosted intraperitoneally or intravenously after an appropriate term from initial immunization. When the amount of an antigen is small, a solution in which the antigen is suspended may be directly injected into a mouse spleen to effect immunization.

After exenterating a spleen and peeling an adipose tissue off after several days from the final immunization, a spleen cell suspension is prepared. The spleen cell is fused, for example, with an HGPRT-deficient myeloma cell to prepare a hybridoma. As a cell fusion agent, any means capable of efficiently fusing a spleen cell (B cell) and a myeloma cell is applicable, and for example, a method of using a hemagglutinating virus of Japan (HVJ), polyethyleneglycol (PEG) and the like are recited. Cell fusion may be conducted by a method using a high voltage pulse.

After the cell fusion operation, cells are cultured in an HAT medium, a clone of a hybridoma in which a spleen cell and a myeloma cell are fused is selected, and the cell is allowed to grow until screening becomes possible. In a method of detecting an antibody for selecting a hybridoma that produces an intended antibody, or a method of measuring a titer of an antibody, an antigen-antibody reaction system may be used. Concretely, as a method of measuring an antibody against a soluble antigen, a radioisotope immune assay (RIA), an enzyme-linked immunosorbent assay (ELISA) and the like can be recited.

Considering the property of the methylated DNA antibody (one antibody binds to one methylated base (cytosine)), it is desired to select a region where a number of methylated bases (cytosine), namely CpG are present as a target DNA region, and thus an improvement in quantification accuracy and detection sensitivity is expected.

The term “specific oligonucleotide” means an oligonucleotide comprising a nucleotide sequence capable of base-pairing with single-stranded DNA containing the target DNA region, and not inhibiting binding of the methylated DNA antibody to a methylated base (cytosine) in single-stranded DNA containing the target DNA region in base-pairing with single-stranded DNA containing the target DNA region. In other words, it is an oligonucleotide that will not inhibit binding between the methylated base in the target DNA region in single-stranded DNA and the methylated DNA antibody. More concretely, it is, for example, an oligonucleotide that will not inhibit binding between methylcytosine in the target DNA region and the methylcytosine antibody, and is capable of binding with single-stranded DNA containing the target DNA region by complementation.

The wording “nucleotide sequence capable of binding with single-stranded DNA containing the target DNA region” means a nucleotide sequence required for forming a bound body (double strand) with single-stranded DNA containing the target DNA region, namely, a nucleotide sequence containing a nucleotide sequence complementary to a part of the nucleotide sequence of the target DNA region, or a nucleotide sequence containing a nucleotide sequence complementary to a part of a nucleotide sequence of a DNA region on a further 5′-end side than 5′-end of the target DNA region, or a nucleotide sequence containing a nucleotide sequence complementary to a part of a nucleotide sequence of a DNA region on a further 3′-end side than 3′-end of the target DNA region. The wording “not inhibiting binding of the methylated DNA antibody to a methylated base (cytosine) in the single-stranded DNA containing the target DNA region” means that the nucleotide sequence of the specific oligonucleotide is such a nucleotide sequence that complementary binding between the specific oligonucleotide and the single-stranded DNA will not occur in an occupied space required for binding of the methylated DNA antibody with methylated single-stranded DNA. That is, it is supposed that for the methylated DNA antibody to bind with the methylated base (cytosine), not only the directly binding methylated base (cytosine), but also a peripheral space where the methylated base (cytosine) exists is also occupied. Therefore, the specific oligonucleotide should be the one that fails to complementarily bind with the single-stranded DNA in the occupied space required for the methylated DNA antibody in binding with the methylated DNA.

The present invention enables simultaneous detection of at least one target DNA region using at least one specific oligonucleotide. That is, the specific oligonucleotide to be bound to the single-stranded DNA is not necessarily one kind, but two or more kinds may be used unless binding of the methylated DNA antibody is inhibited. By using plural kinds of specific oligonucleotides, it is possible to improve the quantification accuracy and detection accuracy. Further, by detecting plural target regions simultaneously, it is possible to increase the amount of methylated DNA to be measured.

As concrete examples of the “specific oligonucleotide”, a nucleotide sequence of any of SEQ ID NOs: 13 to 24, and complementary sequences thereof can be recited, or nucleotide sequences having a sequence identity of 80% or more, 90% or more, 95% or more, or 98% or more with such nucleotide sequences are also recited.

In Second step, as a preferred form in forming the complex of the single-stranded DNA, the methylated DNA antibody, and the specific oligonucleotide, formation in a reaction system containing a divalent cation is recited. More preferably, the divalent cation is a magnesium ion. Here, the “reaction system containing a divalent cation” means such a reaction system that a divalent cation is contained in an annealing buffer used for binding between the single-stranded DNA containing the target DNA region and the specific oligonucleotide, and concretely, for example, a salt composed of a magnesium ion (for example, MgOAc₂, MgCl₂ or the like) may be contained in a concentration of 1 mM to 600 mM.

Besides this, in Second step, as a preferred form in forming the complex of the single-stranded DNA, the methylated DNA antibody and the specific oligonucleotide, a counter oligonucleotide may be added. The counter oligonucleotide is obtained by dividing the same nucleotide sequence as the target DNA region into short oligonucleotides. It may be designed to have usually 10 to 100 bases, and more preferably 20 to 50 bases. The counter oligonucleotide is not designed on the nucleotide sequence where the specific oligonucleotide and the target region bind. The counter oligonucleotide is added in large excess with respect to genomic DNA, and is added for preventing a complementary strand (minus strand) of the target DNA region and a single strand (plus strand) of the target DNA region from rebinding by complementation in causing binding with the specific oligonucleotide (minus strand) after making the target DNA region into the single strand (plus strand). This is because in causing binding of the methylated DNA antibody with the target DNA region, and measuring methylation frequency of DNA and an index value correlated therewith, the target region in a single strand state is more likely to bind with the methylated DNA antibody. The counter oligonucleotide is preferably added in an amount of at least 10 times, and usually 100 times or more compared to the target DNA region.

The wording “formation of a complex” means a mixture in such a condition that the single-stranded DNA containing the methylated target DNA region, the specific oligonucleotide, and the methylated DNA antibody bind. In the present invention, it is preferred to immobilize the complex to a support by binding the methylated DNA antibody or the specific oligonucleotide in the complex with the support before Third step to be described later.

The material and form of the support are not particularly limited as far as a complex can bind thereto. For example, any form suited for use purpose may be employed, including the forms of tube, test plate, filter, disc, bead and so on. As the material, those used as supports for a usual immune measuring method, for example, synthetic resins such as polystyrene, polypropylene, polyacrylamide, polymethylmethacrylate, polysulfone, polyacrylonitrile and nylon, or the synthetic resins incorporating a reactive functional group such as a sulfonic group, or an amino group can be recited. Also, glass, polysaccharides or derivatives thereof (cellulose, nitrocellulose and so on), silica gel, porous ceramics, metal oxides and the like may be used.

As a method of immobilizing the complex to the support by binding, a method of immobilizing the specific oligonucleotide to the support is recited. For binding the single-stranded DNA containing the methylated target DNA region to the support via the specific oligonucleotide, the methylated DNA antibody is quantified or detected by its identification function (to be described later).

For allowing the specific oligonucleotide to be immobilized to the support, the specific oligonucleotide may be immobilized to the support eventually in the condition that the single-stranded DNA containing the methylated target DNA region and the methylated DNA antibody form a complex, and (1) the specific oligonucleotide may be immobilized to the support in the stage before binding of the single-stranded DNA and the specific oligonucleotide, or (2) the specific oligonucleotide may be immobilized to the support in the stage after binding of the single-stranded DNA and the specific oligonucleotide.

For immobilizing a specific oligonucleotide to a support, concretely a method of immobilizing a biotinylated oligonucleotide obtained by biotinylating 5′-end or 3′-end of the specific oligonucleotide to a support coated with streptavidin (for example, a PCR tube coated with streptavidin, magnetic beads coated with streptavidin, a chromatostrip partially coated with streptavidin, or the like) is recited.

Also there is a method of letting 5′-end or 3′-end of a specific oligonucleotide covalently bind with a molecule having an active functional group such as an amino group, a thiol group, or an aldehyde group, and then letting it covalently bind to a support made of glass, a polysaccharide derivative, silica gel, the synthetic resin or thermostable plastic whose surface is activated by a silane coupling agent or the like. Covalent binding is achieved, for example, by a spacer formed by serially connecting five triglycerides, a cross linker or the like. Also there is a method of chemical synthesis from the terminal side of the specific oligonucleotide directly on a support of glass or silicon.

Other concrete methods may be practiced in the following manners, using “biotinylated specific oligonucleotide” whose terminal end is labeled with biotin as a specific oligonucleotide that is immobilizable to a support. (a) A mixture is obtained by adding an annealing buffer (for example, 33 mM Tris-Acetate pH 7.9, 66 mM KOAc, 10 mM MgOAc₂, 0.5 mM Dithiothreitol) and a biotinylated specific oligonucleotide to the DNA sample derived from genomic DNA contained in the biological specimen. Then the obtained mixture is heated at 95° C., for example, for several minutes to make double-stranded DNA derived from genomic DNA contained in the biological specimen into single-stranded DNA. Then the mixture is rapidly cooled to a temperature that is lower than the Tm value of the biotinylated specific oligonucleotide by about 10 to 20° C., and kept at this temperature, for example, for several minutes, and then the temperature is returned to room temperature for allowing formation of a bound body between the single-stranded DNA containing the target DNA region and the biotinylated specific oligonucleotide (the bound body formed in this stage includes a bound body between single-stranded DNA containing an unmethylated target DNA region and the specific oligonucleotide, as well as a bound body between single-stranded DNA containing the methylated target DNA region and the specific oligonucleotide). (b) By adding the mixture obtained in the above (a) to a support coated with streptavidin, and retaining it at 37° C. for example, for several minutes, the bound body between the single-stranded DNA containing the target DNA region and the biotinylated specific oligonucleotide is immobilized to the support coated with streptavidin. Thereafter, the remaining solution is removed and washing is performed. A washing buffer (for example, a 0.05% Tween 20-containing phosphate buffer (1 mM KH₂PO₄, 3 mM Na₂HPO.7H₂O, 154 mM NaCl pH 7.4)) is added, for example, in a proportion of 300 μL/well, and the solution is removed. This washing operation is repeated several times, to leave the bound body between the biotinylated specific oligonucleotide immobilized to the support, and the single-stranded DNA containing the target DNA region, on the well (also in this stage, the bound body formed in this state includes a bound body between single-stranded DNA containing an unmethylated target DNA region and the specific oligonucleotide, as well as a bound body between single-stranded DNA containing the methylated target DNA region and the specific oligonucleotide).

(c) An appropriate amount (for example, 100 μL/well of a 4 μg/mL solution) of a methylated DNA antibody is added to the well, and then left still at room temperature, for example, for about 3 hours, to promote formation of a complex of the methylated DNA antibody, single-stranded DNA containing the methylated target DNA region, of the single-stranded DNA, and the biotinylated specific oligonucleotide (formation of a complex) (in this stage, the bound body between the single-stranded DNA containing an unmethylated target DNA region and the specific oligonucleotide fails to form a complex). Thereafter, the remaining solution is removed and washing is performed. A washing buffer (for example, a 0.05% Tween 20-containing phosphate buffer (1 mM KH₂PO₄, 3 mM Na₂HPO.7H₂O, 154 mM NaCl pH 7.4)) is added, for example, in a proportion of 300 μL/well, and the solution is removed. This washing operation is repeated several times, to leave the complex on the well (selection of a complex).

The annealing buffer used in (a) is not limited to the annealing buffer described above as far as it is suited for binding between the specific oligonucleotide and single-stranded DNA containing the target DNA region derived from a biological sample. Use of a buffer in which a divalent ion, preferably a magnesium ion is dissolved in a concentration of 1 to 600 mM will improve the binding stability.

The washing operation in (b) and (c) is important for removing the methylated DNA antibody that is not immobilized and suspended in the solution, single-stranded DNA that is not bound to the methylated DNA antibody and suspended in the solution, or DNA that has been digested with a restriction enzyme to be described later and suspended in the solution, from the reaction solution. The washing buffer is not limited to the washing buffer described above, insofar as it is suited for removing the free methylated DNA antibody, single-stranded DNA and so on suspended in the solution, and a DELFIA buffer (available from PerkinElmer, Tris-HCl pH 7.8 with Tween 80), a TE buffer and the like may be used.

As described above, in the above (a) to (c), binding between single-stranded DNA containing the target DNA region and the biotinylated specific oligonucleotide is conducted in the stage previous to the immobilization of the biotinylated specific oligonucleotide to the support coated with streptavidin, however, this order may be inverted. In other words, for example, a mixture is obtained by adding the DNA sample derived from genomic DNA contained in the biological specimen to the biotinylated specific oligonucleotide immobilized to the support coated with streptavidin. For making double-stranded DNA containing the target DNA region possessed by genomic DNA contained in the biological specimen into a single strand, the obtained mixture is heated at 95° C., for example, for several minutes, and then for allowing formation of a bound body with the biotinylated specific oligonucleotide, the reaction is rapidly cooled to a temperature lower than the Tm value of the biotinylated specific oligonucleotide by about 10 to 20° C., and retained at this temperature, for example, for several minutes (the bound body formed in this stage includes a bound body between methylated single-stranded DNA other than the target DNA region and the methylated antibody, as well as a bound body between single-stranded DNA containing the methylated target DNA region and the methylated antibody). Thereafter, the operation of (c) may be practiced, to form and select a complex (in this stage, a bound body between single-stranded DNA containing an unmethylated target DNA region and the specific oligonucleotide fails to form a complex).

The operations of the above (a) to (c) may be conducted using a chromatostrip. In this case, the operation is conducted in the following manner. The solution in which the bound body between the single-stranded DNA containing the target DNA region possessed by genomic DNA contained in the biological specimen and the biotinylated specific oligonucleotide is formed is developed by a chromatostrip partially coated with streptavidin. By this operation, the bound body between the single-stranded DNA containing the target DNA region possessed by genomic DNA contained in the biological specimen and the biotinylated specific oligonucleotide is immobilized to the part coated with streptavidin (the bound body formed in this stage includes a bound body between methylated single-stranded DNA other than the target DNA region and the methylated antibody, as well as a bound body between single-stranded DNA containing the methylated target DNA region and the methylated antibody). Then an appropriate amount of the methylated DNA antibody is developed by a chromatostrip as described above. Through these operations, the complex of single-stranded DNA containing the methylated target DNA region possessed by genomic DNA contained the biological specimen, the biotinylated specific oligonucleotide, and the methylated DNA antibody is immobilized to the part coated with streptavidin (formation and selection of a complex) (in this stage, the bound body between the single-stranded DNA containing an unmethylated target DNA region and the specific oligonucleotide fails to form a complex). Also for these operations, the order of formation of a complex may be inverted. For example, after forming a complex made up of single-stranded DNA containing the methylated target DNA region, a biotinylated specific oligonucleotide, and a methylated DNA antibody, the complex may be developed by a chromatostrip, and immobilized to the part coated with streptavidin. In these operations, unnecessary components can be removed by developing the solution by a chromatostrip, and a washing operation can be omitted. Of course, a washing operation (development of a chromatostrip by a washing buffer (for example, a 0.05% Tween 20-containing phosphate buffer (1 mM KH₂PO₄, 3 mM Na₂HPO.7H₂O, 154 mM NaCl pH 7.4))) may be conducted between operations without causing any problem.

The “identification function” in Third step is a function based on labeling used for detection or quantification, and concretely, in the case of a methylated DNA antibody, a fluorescent or a chromogenic property of a label in a methylated DNA antibody labeled with europium, gold colloid, latex beads, a radioisotope, a fluorescent substance (FITC or the like), horseradish Peroxidase (HRP), alkaline phosphatase, biotin or the like can be recited, and the property that the antibody itself binds to a secondary antibody and the like are also recited as the function. As a means for quantifying or detecting such a function, for example, measurement by a radiation detector or a spectrophotometer, or visual check may be used.

In the case of a methylated DNA antibody, as described above, the property that the antibody itself binds to a secondary antibody or the like is considered as the function. That is, even when the antibody itself is not labeled, a methylated DNA antibody can be indirectly quantified or detected by using a secondary antibody to which the quantifiable or detectable recognition function is imparted.

When a methylated DNA antibody is quantified or detected depending on its identification function, concretely, when the property of the antibody itself is used as the function, the operation may be conducted in the following manner. A secondary antibody against a methylated DNA antibody (for example, an Eu-N1-labeled mouse IgG antibody: available from PerkinElmer) is added to a complex, and left still at room temperature for about an hour, to promote binding of the secondary antibody to the complex. Then, Enhancement Solution (available from PerkinElmer) is added and mixed, and left still, for example, for about 45 minutes at room temperature. Thereafter, the fluorescence (excitation 340 nm/fluorescence 612 nm) is measured by a fluorescence detector to quantify or detect the methylated DNA antibody. When an antibody to which FITC is bound is used as a secondary antibody, the methylated DNA antibody may be quantified or detected by measuring fluorescence of FITC by a known method. For example, when biotin is not used for immobilization of the specific oligonucleotide, a biotinylated methylated DNA antibody may be used for quantification or detection. When the biotinylated methylated DNA antibody is quantified or detected, a biotinylated methylated DNA antibody can be quantified or detected, for example, by adding and mixing HRP-labeled streptavidin with the biotinylated methylated DNA antibody, and measuring the activity of HRP by a known method after formation of a bound body between the biotinylated methylated DNA antibody and HRP-labeled streptavidin.

The present invention includes a modified method (Modified method 1) additionally comprising a step of digesting unmethylated single-stranded DNA by a methylation sensitive restriction enzyme capable of digesting single-stranded DNA after First step and before Third step, and a modified method (Modified method 2) additionally comprising a step of digesting an unmethylated single-stranded DNA by a methylation sensitive restriction enzyme in a reaction system containing a specific masking oligonucleotide comprising a recognition sequence for the methylation sensitive restriction enzyme as its part after First step and before Third step of the present invention.

The “methylation sensitive restriction enzyme” in these modified methods (Modified method 1 and Modified method 2) means, for example, a restriction enzyme or the like that fails to digest a recognition sequence containing methylated cytosine, but digests only a recognition sequence containing unmethylated cytosine. In other words, in the case of DNA wherein cytosine contained in a recognition sequence inherently recognizable by the methylation sensitive restriction enzyme is methylated, the DNA will not be cleaved even when the methylation sensitive restriction enzyme is caused to act on the DNA. On the other hand, in the case of DNA wherein cytosine contained in a recognition sequence inherently recognizable by the methylation sensitive restriction enzyme is not methylated, the DNA will be cleaved when the methylation sensitive restriction enzyme is caused to act on the DNA. Concrete examples of such methylation sensitive enzymes include HpaII, BstUI, NarI, SacII, and HhaI. The methylation sensitive restriction enzyme fails to cleave double-stranded DNA in a hemimethyl state (double-stranded DNA wherein cytosine (CpG) in one strand is methylated, and complementary cytosine (CpG) in the other strand is not methylated), and this is already revealed by Gruenbaum et al. (Nucleic Acid Research, 9, 2509-2515).

The wording “methylation sensitive restriction enzyme capable of digesting single-stranded DNA” means, for example, a restriction enzyme or the like capable of digesting only a recognition sequence containing unmethylated cytosine without digesting a recognition sequence containing methylated cytosine in single-stranded DNA. In other words, some methylation sensitive restriction enzymes digest single-stranded DNA. For example, HhaI or the like is recited.

As a fear in a digestion treatment with a methylation sensitive restriction enzyme, the possibility that the recognition sequence containing unmethylated cytosine may not be completely digested (so called “DNA remaining undigested”) is recited. When such a possibility is problematic, the target DNA region has at least one recognition site of a methylation sensitive restriction enzyme, and as many as possible recognition sites would be preferred because it is possible to suppress the “DNA remaining undigested” as much as possible by presence of abundant recognition sites of a methylation sensitive restriction enzyme.

As described above, as to the target DNA region, from the viewpoint of the property of the methylated DNA antibody (one antibody binds to one methylated base (cytosine)), a region where a number of methylated bases (cytosine, CpG) exist is desirably selected as a target DNA region, and from the viewpoint of minimizing “DNA remaining undigested”, a region where a number of recognition sites of a methylation sensitive restriction enzyme exist is desirably selected.

The term “specific masking oligonucleotide” in Modified method 2 means an oligonucleotide comprising a recognition sequence for a methylation sensitive restriction enzyme as its part. Concretely, it is an oligonucleotide that forms double-stranded DNA by complementary binding with one specific site (one or more sites may be concurrently contained in the following 8 to 30 bases depending on the nucleotide sequence of the target region) of several recognition sequences of a methylation sensitive restriction enzyme contained in the target DNA region in single-stranded DNA (namely, the site is made into a double-stranded state), thereby enabling the methylation sensitive restriction enzyme that uses only double-stranded DNA as a substrate to digest the site, and improving digestion efficiency at the site for the methylation sensitive restriction enzyme capable of digesting single-stranded DNA (the methylation sensitive restriction enzyme capable of digesting single-stranded DNA also digests double-stranded DNA, and digestion efficiency thereof is higher for double-stranded DNA than for single-stranded DNA), and is a nucleotide not inhibiting formation of a bound body between single-stranded DNA containing DNA of a target region and the specific oligonucleotide.

As the specific masking oligonucleotide, more concretely, oligonucleotides comprising a nucleotide sequence complementary to a nucleotide sequence having a length of 8 to 30 bases containing one specific site (one or more sites may be concurrently contained in the following 8 to 30 bases depending on the nucleotide sequence of the target) among several recognition sequences of the methylation sensitive restriction enzyme contained in the target DNA region in single-stranded DNA, and not inhibiting formation of the bound body between the single-stranded DNA containing DNA of the target region and the specific oligonucleotide can be recited.

The specific masking oligonucleotide to be mixed with the DNA sample derived from genomic DNA may be one kind or plural kinds. When plural kinds are used, many of recognition sites of the methylation sensitive restriction enzyme in single-stranded DNA containing a target DNA region become a double-stranded DNA state, and “DNA remaining undigested” as described above by a methylation sensitive restriction enzyme can be minimized. However, since the methylated DNA antibody is no longer able to bind to the site where the specific masking oligonucleotide forms double-stranded DNA, it is not appropriate to use the specific masking oligonucleotide for too many recognition sites of the methylation sensitive restriction enzyme, and it is preferred to use appropriate kinds. If the specific masking oligonucleotide is used for every recognition site of the methylation sensitive restriction enzyme contained in the target DNA region, binding between a methylated base (cytosine) and the methylated DNA antibody in the recognition site is inhibited, and the probability of formation of a complex comprising a methylated target DNA region can be decreased (of course, the methylated DNA antibody binds to CpG other than the recognition site, however, when the methylated DNA antibody is quantified or detected, the quantification accuracy and the detection sensitivity are decreased). For example, it is particularly useful to use the specific masking oligonucleotide designed for the site where it is necessarily intended not to be digested when it is methylated and intended to be digested when it is not methylated among several recognition sequences of the methylation sensitive restriction enzyme contained in the target DNA region (for example, the site that is methylated at 100% in an affected diseased patient specimen, but is not methylated at 100% in a healthy subject specimen).

In these Modified methods (Modified method 1 and Modified method 2), by conducting the step of digesting by at least one kind of methylation sensitive restriction enzyme after First step and before Third step, it is possible to minimize (exclude) formation of a complex comprising an unmethylated target DNA region, so that the quantification accuracy and the detection sensitivity of methylated DNA in the target DNA region are improved.

In Modified method 1, the operation of digesting the single-stranded DNA obtained in First step by at least one kind of methylation sensitive restriction enzyme capable of digesting single-stranded DNA executed after end of First step and before start of Third step may be concretely executed in the following manner. To 10 μL of a specimen solution containing single-stranded DNA obtained in First step, 3 μL of an optimum 10× buffer, 15 U of a methylation sensitive enzyme (HhaI) capable of digesting a single strand, and an appropriate amount of BSA or the like as necessary are added, and then the resultant mixture is added with sterile ultrapure water to a liquid volume of 30 μL, and then incubated at 37° C., for example, for an hour to overnight. As a result, the HhaI recognition site unmethylated in the target DNA region will be digested (a treatment solution). For example, when this operation is conducted before formation of a complex, the treatment solution may be directly used for formation of a complex because a washing operation is executed at the time of formation of a complex and (or) selection. When this operation is conducted after selection of a complex, since it is necessary to remove single-stranded DNA (digested and generated single-stranded DNA because the recognition site of the methylation sensitive restriction enzyme is not methylated) suspended in the treatment solution from the treatment solution, it is necessary to conduct a washing operation similar to that executed in formation of a complex and (or) selection again after end of this operation.

In Modified method 2, the operation of conducting a digestion treatment by adding the single-stranded DNA obtained in First step, with at least one kind of methylation sensitive restriction enzyme and the specific masking oligonucleotide comprising a recognition sequence for the methylation sensitive restriction enzyme as its part, after end of First step and before start of Third step, may be concretely conducted, for example, in the following manner. To 10 μL of a specimen solution containing single-stranded DNA obtained in First step, 3 μL of an optimum 10× buffer, 15 U of a methylation sensitive enzyme (HhaI, HhaI or the like), about 10 pmol of the specific masking oligonucleotide for one specific site among recognition sequences of the methylation sensitive enzyme, and an appropriate amount of BSA or the like as necessary are added, and then the resultant mixture is added with sterile ultrapure water to a liquid volume of 30 μL, and then incubated at 37° C., for example, for an hour to overnight. As a result, the site unmethylated in the target DNA region will be digested (a treatment solution). For example, when this operation is conducted before formation of a complex, the treatment solution may be directly used for formation of a complex because the washing operation is conducted at the time of formation of a complex and (or) selection. When this operation is conducted after selection of a complex, since it is necessary to remove single-stranded DNA (digested and generated single-stranded DNA because a recognition site of the methylation sensitive restriction enzyme is not methylated) suspended in the treatment solution from the treatment solution, it is necessary to conduct a washing operation similar to that executed in formation of a complex and (or) selection again after end of this operation.

In the present invention, in one preferred embodiment, “a DNA sample derived from genomic DNA contained in the biological specimen” is a DNA sample that is preliminarily digested with a restriction enzyme recognition cleaving site for which is not present in the target DNA region of the genomic DNA. In forming a bound body between single-stranded DNA containing the target DNA region possessed by genomic DNA contained in the biological specimen and the specific oligonucleotide, it is expected that the shorter the single-stranded DNA is, the better the operability is and the more easily a complex is formed as far as the target DNA region is contained. To shorten the single-stranded DNA, it is efficient to make it short when it is in former genomic DNA. Therefore, a digestion treatment may be preliminarily conducted using a restriction enzyme recognition cleaving site for which is not present in the target DNA region on a DNA sample derived from genomic DNA contained in the biological specimen. As a method of the digestion treatment by the restriction enzyme whose recognition cleaving site is not present in the target DNA region, a generally known restriction enzyme treatment method may be used. When the specimen is a DNA sample preliminarily purified, a digestion treatment may be executed using a generally used amount of a restriction enzyme, whereas when the specimen is a tissue lysate, a cell lysate or the like, a digestion treatment may be conducted using a large excess of a methylation sensitive restriction enzyme, for example, an amount of 500 times or more the DNA amount of a restriction enzyme.

In one preferred embodiment, “a DNA sample derived from genomic DNA contained in the biological specimen” is a DNA sample that has been digested with at least one kind of methylation sensitive restriction enzyme. By preliminarily digesting a biological specimen itself with a methylation sensitive restriction enzyme, it is possible to quantify or detect methylation at high accuracy in Final step. This method is useful for eliminating “DNA remaining undigested” as described above. As a method of digesting the specimen itself with a methylation sensitive restriction enzyme, when genomic DNA in the specimen exists in a state of double-stranded DNA, a generally known restriction enzyme treatment may be conducted using a methylation sensitive restriction enzyme. On the other hand, when genomic DNA in the specimen exists in a state of single-stranded DNA or when genomic DNA in the specimen exists in combined states of double-stranded DNA and single-stranded DNA, a digestion treatment may be conducted by the method corresponding to Modified method 1 or Modified method 2. Concretely, for example, to 10 μL of a specimen solution, 5 μL of an optimum 10× buffer, 15 U of a methylation sensitive restriction enzyme (HhaI) capable of digesting single-stranded DNA, and an appropriate amount of BSA or the like as necessary are added, and then the resultant mixture is added with sterile ultrapure water to a liquid volume of 50 μL, and then incubated at 37° C., for example, for an hour to overnight. Alternatively, to 10 μL of a specimen, 5 μL of an optimum 10× buffer, 15 U of a methylation sensitive restriction enzyme (HpaII, HhaI or the like), about 15 pmol of a specific masking oligonucleotide for one specific site among recognition sequences of the methylation sensitive enzyme, and an appropriate amount of BSA or the like as necessary are added and then the resultant mixture is added with sterile ultrapure water to a liquid volume of 50 μL, and then incubated at 37° C., for example, for an hour to overnight. When the specimen is a preliminarily purified DNA sample, the treatment may be executed using a generally used amount of a restriction enzyme, whereas when the specimen is a tissue lysate, a cell lysate or the like, the treatment may be conducted using a large excess of a methylation sensitive restriction enzyme, for example, an amount of 500 times or more the DNA amount of a methylation sensitive restriction enzyme.

As a method of quantifying or detecting minor substances contained in a biological sample such as blood or urine, immunological measuring methods are generally used. Among such methods, a so-called immunochromatography using chromatography is widely used in various situations including, for example, clinical examinations in hospitals, assays in laboratories and so on because of its simple operation and short time required for assay. In recent years, a so-called hybrid chromatography has been utilized in which labeled DNA (gene) is developed on a chromatostrip, and target DNA (gene) is detected by hybridization using a probe capable of capturing the target DNA (gene). Also this method is now coming to be widely used in situations including, for example, clinical examinations in hospitals, assays in laboratories and so on because of its simple operation and short time required for assay. The present invention conceptually enables a combined method of the immunochromatography and the hybrid chromatography. In the present invention, since the order of formation of a complex and selection of a complex is not particularly limited, various methods are possible. Concretely, such methods may be executed in the following manner.

Method 1: A sample after end of First step is added with the biotinylated specific oligonucleotide, to cause formation of a bound body between single-stranded DNA containing the target DNA region and the biotinylated specific oligonucleotide (the bound body formed in this stage includes not only a bound body between single-stranded DNA containing the methylated target DNA region and the specific oligonucleotide, but also a bound body between single-stranded DNA containing an unmethylated target DNA region and the specific oligonucleotide), and added with a methylated antibody having the function, to cause formation of a complex of the single-stranded DNA containing the methylated target DNA region, the biotinylated specific oligonucleotide, and a methylated DNA antibody having the function (in this stage, the bound body between the single-stranded DNA containing an unmethylated target DNA region and the specific oligonucleotide fails to form a complex). Upon dropping (introduction) of the obtained sample into an introduction part of a chromatostrip, the complex migrates in a development part by a capillary phenomenon, and is trapped in the part preliminarily coated with streptavidin. Thereafter, by quantifying or detecting the methylated DNA antibody forming the obtained complex according to its function, methylated DNA in the target DNA region can be quantified or detected.

Method 2: A sample after end of First step is added with the biotinylated specific oligonucleotide, to cause formation of a bound body between single-stranded DNA containing the target DNA region and the biotinylated specific oligonucleotide (the bound body formed in this stage includes not only a bound body between single-stranded DNA containing the methylated target DNA region and the specific oligonucleotide, but also a bound body between single-stranded DNA containing an unmethylated target DNA region and the specific oligonucleotide). Upon dropping (introduction) of the obtained sample into an introduction part of a chromatostrip, the bound body migrates in a development part by a capillary phenomenon, and is trapped in the part preliminarily coated with streptavidin (also in this stage, the bound body includes not only a bound body between single-stranded DNA containing the methylated target DNA region and the specific oligonucleotide, but also a bound body between single-stranded DNA containing an unmethylated target DNA region and the specific oligonucleotide). Then, upon dropping (introduction) of a methylated antibody having the function into an introduction part, it migrates in a development part and binds to methylated cytosine of the bound body, to form a complex of single-stranded DNA containing the methylated target DNA region, the biotinylated specific oligonucleotide, and a methylated DNA antibody having the function (in this stage, the bound body between the single-stranded DNA containing an unmethylated target DNA region and the specific oligonucleotide fails to form a complex). By quantifying or detecting a methylated DNA antibody contained in the obtained complex according to its function, methylated DNA in the target DNA region can be quantified or detected.

Method 3: Upon dropping (introduction) of the biotinylated specific oligonucleotide into an introduction part of a chromatostrip, the oligonucleotide migrates in a development part by a capillary phenomenon, and is trapped in the part preliminarily coated with streptavidin. Then, upon dropping (introduction) of a sample after end of First step into an introduction part, it migrates in a development part, and is trapped by the biotinylated specific oligonucleotide that has been already trapped in the condition that single-stranded DNA containing the target DNA region forms a bound body (the bound body formed in this stage includes not only a bound body between single-stranded DNA containing the methylated target DNA region and the specific oligonucleotide, but also a bound body between single-stranded DNA containing an unmethylated target DNA region and the specific oligonucleotide). Then, upon dropping (introduction) of a methylated antibody having the function into an introduction part, it migrates in a development part, and binds to methylated cytosine of the bound body, to form a complex of single-stranded DNA containing the methylated target DNA region, the biotinylated specific oligonucleotide, and the methylated DNA antibody having the function (in this stage, the bound body between the single-stranded DNA containing an unmethylated target DNA region and the specific oligonucleotide fails to form a complex). By quantifying or detecting the methylated DNA antibody forming the obtained complex according to its function, methylated DNA in the target DNA region can be quantified or detected.

Method 4: Upon dropping (introduction) of the biotinylated specific oligonucleotide into an introduction part of a chromatostrip, the oligonucleotide migrates in a development part by a capillary phenomenon, and is trapped in the part preliminarily coated with streptavidin. A sample after end of First step is added with the methylated DNA antibody having the function, to form a bound body between single-stranded DNA having methylated cytosine (in which single-stranded DNA containing the target DNA region and single-stranded DNA other than the target exist) and the methylated DNA antibody having the function (the bound body formed in this stage includes not only a bound body between single-stranded DNA containing the methylated target DNA region and the methylated antibody, but also a bound body between methylated single-stranded DNA other than the target DNA region and the methylated antibody). Upon dropping (introduction) of the obtained bound body into an introduction part, it migrates in a development part, and single-stranded DNA containing the methylated target DNA region binds to the biotinylated specific oligonucleotide that has been already trapped, to form a complex of single-stranded DNA containing the methylated target DNA region, the biotinylated specific oligonucleotide, and the methylated DNA antibody having the function (in this stage, the bound body between methylated single-stranded DNA other than in the target DNA region and a methylated antibody fails to form a complex). By quantifying or detecting the methylated DNA antibody contained in the obtained complex according to its function, methylated DNA in the target DNA region can be quantified or detected.

Also a plurality of detection sites may be provided on a single chromatostrip (the specific oligonucleotides capable of trapping different target DNA regions are immobilized to a support), and each target DNA region may be sequentially quantified or detected, and by enabling one detection site to trap a plurality of target DNA regions, or by immobilizing a number of the specific oligonucleotides capable of trapping a plurality of target DNA regions on one detection site, it is possible to dramatically improve the detection sensitivity. Also, using a number of the specific oligonucleotides having the function capable of trapping a plurality of target DNA regions so as to allow formation of a complex with a plurality of target DNA regions will also improve the detection sensitivity dramatically. Further, designing a number of the specific oligonucleotides in a single target region, and using these on the side of a support or on the side of detection will also improve the detection sensitivity dramatically.

The restriction enzyme, the specific oligonucleotide, or the methylated DNA antibody which may be used in the present invention as described above is useful as a reagent in a detection kit. The present invention also provides a detection kit containing such a restriction enzyme, the specific oligonucleotide, or the methylated DNA antibody and so on as a regent, and a detection chip including a support on which the specific oligonucleotide, or the methylated DNA antibody and so on is immobilized, and the scope of the present invention implies use in the form of a detection kit or a detection chip as described above utilizing a substantial principle of the method.

### Examples

In the following, the present invention will be explained in detail by way of examples, which are not intended to limit the present invention.

### Example 1

A colon-adenocarcinoma cell strain Capan-2 (ATCC NO. HTB-80) derived from a mammal and purchased from ATCC was cultured to be confluent in a culture medium in which equivalent amounts of McCoy's5a (Gibco, Cat. No. 16600-082) and FBS (ATCC, CatNo. 30-2020) were mixed, to obtain about 1×10⁷ of cells. The obtained cells were added with a SEDTA buffer [10 mM Tris-HCl pH 8.0, 10 mM EDTA pH 8.0, 100 mM NaCl] in a volume of 10 times, and homogenized.

The obtained mixture was added with proteinase K (Sigma) in a concentration of 500 μg/mL and sodium dodecyl sulfate in a concentration of 1% (w/v), and shaken at 55° C. for about 16 hours, and then subjected to a phenol/chloroform extraction treatment using phenol saturated with 1 M Tris-HCl, pH 8.0. The aqueous layer was collected, and added with NaCl in a concentration of 0.5 N, and the precipitate (genomic DNA) generated by an ethanol precipitation treatment was collected. The collected precipitate was dissolved in a TE buffer (10 mM Tris, 1 mM EDTA, pH 8.0), and added with RNase A (Sigma) in a concentration of 40 μg/ml, and incubated at 37° C. for 1 hour, and then subjected to a phenol/chloroform extraction treatment. The aqueous layer was collected, added with NaCl in a concentration of 0.5 N, and the precipitate (genomic DNA) generated by an ethanol precipitation treatment was collected. The collected precipitate was rinsed with 70% ethanol, to obtain Capan-2 genomic DNA.

Each of 500 ng/20 μL solutions in TE buffer of Genomic DNA derived from human blood purchased from Clontech, and the above-described Capan-2 genomic DNA was prepared in duplicate.

Each 20 μL of the prepared genomic DNA solution, 10 U of restriction enzyme XspI, and 5 μL of 10× buffer optimum for XspI (200 mM Tris-HCl pH 8.5, 100 mM MgCl₂, 10 mM Dithiothreitol, 1000 mM KCl) were mixed, and added with sterilized ultrapure water to make the liquid volume 50 μL, and the liquid was incubated at 37° C. for 1 hour, to prepare a genome solution.

As a specific oligonucleotide used for obtaining a target DNA region of either of SEQ ID NOs: 1 to 12, 5′-end biotinylated oligonucleotide having a nucleotide sequence of SEQ ID NOs: 13 to 24, and biding to the DNA region by complementation was synthesized, and each 0.1 pmoL/μL solution in TE buffer was prepared in the following manner.

A solution of 5′-end biotinylated oligonucleotide having a nucleotide sequence of SEQ ID NO: 13 for obtaining a target region of SEQ ID NO: 1 was named a specific oligonucleotide solution 1.

A solution of 5′-end biotinylated oligonucleotide having a nucleotide sequence of SEQ ID NO: 13 for obtaining a target region of SEQ ID NO: 1 was named a specific oligonucleotide solution 1.

A solution of 5′-end biotinylated oligonucleotide having a nucleotide sequence of SEQ ID NO: 14 for obtaining a target region of SEQ ID NO: 2 was named a specific oligonucleotide solution 2.

A solution of 5′-end biotinylated oligonucleotide having a nucleotide sequence of SEQ ID NO: 15 for obtaining a target region of SEQ ID NO: 3 was named a specific oligonucleotide solution 3.

A solution of 5′-end biotinylated oligonucleotide having a nucleotide sequence of SEQ ID NO: 16 for obtaining a target region of SEQ ID NO: 4 was named a specific oligonucleotide solution 4.

A solution of 5′-end biotinylated oligonucleotide having a nucleotide sequence of SEQ ID NO: 17 for obtaining a target region of SEQ ID NO: 5 was named a specific oligonucleotide solution 5.

A solution of 5′-end biotinylated oligonucleotide having a nucleotide sequence of SEQ ID NO: 18 for obtaining a target region of SEQ ID NO: 6 was named a specific oligonucleotide solution 6.

A solution of 5′-end biotinylated oligonucleotide having a nucleotide sequence of SEQ ID NO: 19 for obtaining a target region of SEQ ID NO: 7 was named a specific oligonucleotide solution 7.

A solution of 5′-end biotinylated oligonucleotide having a nucleotide sequence of SEQ ID NO: 20 for obtaining a target region of SEQ ID NO: 8 was named a specific oligonucleotide solution 8.

A solution of 5′-end biotinylated oligonucleotide having a nucleotide sequence of SEQ ID NO: 21 for obtaining a target region of SEQ ID NO: 9 was named a specific oligonucleotide solution 9.

A solution of 5′-end biotinylated oligonucleotide having a nucleotide sequence of SEQ ID NO: 22 for obtaining a target region of SEQ ID NO: 10 was named a specific oligonucleotide solution 10.

A solution of 5′-end biotinylated oligonucleotide having a nucleotide sequence of SEQ ID NO: 23 for obtaining a target region of SEQ ID NO: 11 was named a specific oligonucleotide solution 11.

A solution of 5′-end biotinylated oligonucleotide having a nucleotide sequence of SEQ ID NO: 24 for obtaining a target region of SEQ ID NO: 12 was named a specific oligonucleotide solution 12.

Equivalent amounts of the specific oligonucleotide solutions 1 to 12 were mixed, to prepare a specific oligonucleotide solution 13.

As a counter oligonucleotide designed with respect to the nucleotide sequence in the target region of either of SEQ ID NOs: 1 to 12, an oligonucleotide of either of SEQ ID NOs: 25 to 118 was synthesized, and respective 10 pmol/μL solutions in TE buffer were prepared. Equivalent amounts of the counter oligonucleotide solutions targeting the same DNA region were mixed, to prepare respective counter oligonucleotide mixed solutions shown below.

A mixed solution of oligonucleotides of SEQ ID NOs: 25 to 42 was named a counter oligonucleotide mixed solution 1.

A mixed solution of oligonucleotides of SEQ ID NOs: 43 to 60 was named a counter oligonucleotide mixed solution 2.

A mixed solution of oligonucleotides of SEQ ID NOs: 61 to 66 was named a counter oligonucleotide mixed solution 3.

A mixed solution of oligonucleotides of SEQ ID NOs: 67 to 70 was named a counter oligonucleotide mixed solution 4.

A mixed solution of oligonucleotides of SEQ ID NOs: 71 to 76 was named a counter oligonucleotide mixed solution 5.

A mixed solution of oligonucleotides of SEQ ID NOs: 77 to 82 was named a counter oligonucleotide mixed solution 6.

A mixed solution of oligonucleotides of SEQ ID NOs: 83 to 88 was named a counter oligonucleotide mixed solution 7.

A mixed solution of oligonucleotides of SEQ ID NOs: 89 to 94 was named a counter oligonucleotide mixed solution 8.

A mixed solution of oligonucleotides of SEQ ID NOs: 95 to 100 was named a counter oligonucleotide mixed solution 9.

A mixed solution of oligonucleotides of SEQ ID NOs: 101 to 106 was named a counter oligonucleotide mixed solution 10.

A mixed solution of oligonucleotides of SEQ ID NOs: 107 to 112 was named a counter oligonucleotide mixed solution 11.

A mixed solution of oligonucleotides of SEQ ID NOs: 113 to 118 was named a counter oligonucleotide mixed solution 12.

Equivalent amounts of the above counter oligonucleotide mixed solutions 1 to 12 were mixed, to prepare a counter oligonucleotide mixed solution 13.

Equivalent amounts of a specific oligonucleotide solution and a counter oligonucleotide mixed solution were mixed, to prepare the following Solution A, Solution B and Solution C.

Solution A: specific oligonucleotide solution 1 and counter oligonucleotide mixed solution 1

Solution B: specific oligonucleotide solution 2 and counter oligonucleotide mixed solution 2

Solution C: specific oligonucleotide solution 13 and counter oligonucleotide mixed solution 13

In a PCR tube, 30 μL of the genome solution prepared as described above, 30 μL of either one of the Solutions A to C, 15 μL of a buffer (330 mM Tris-Acetate pH 7.9, 660 mM KOAc, 100 mM MgOAc₂, 5 mM Dithiothreitol), 60 μL of a 10 mM MgCl₂ solution, and 15 μL of a 1 mg/mL BSA solution were added, to make the liquid volume 150 μL, and mixed. Then, the PCR tube was heated at 95° C. for 10 minutes, rapidly cooled to 70° C., and retained for 10 minutes at that temperature. Then the reaction was cooled to 50° C. and retained for 10 minutes, and further retained at 37° C. for 10 minutes, and then the reaction was returned to room temperature, to promote formation of a bound body between the 5′-end biotin-labeled oligonucleotide and the DNA fragment.

All the obtained mixtures were transferred to wells coated with streptavidin, and left still for about 30 minutes at room temperature, to immobilize the bound bodies between the 5′-end biotin-labeled oligonucleotides and the DNA fragments to wells. Thereafter, the solution was removed by pipetting, and each well was washed with 200 μL of a washing buffer [0.05% Tween 20-containing phosphate buffer (1 mM KH₂PO₄, 3 mM Na₂HPO.7H₂O, 154 mM NaCl pH 7.4)] three times.

Each well was added with 100 μL of a methylcytosine antibody [available from Aviva Systems Biology, 0.5 μg/mL 0.1% BSA-containing phosphate buffer (1 mM KH₂PO₄, 3 mM Na₂HPO.7H₂O, 154 mM NaCl pH 7.4) solution], and left still for 1 hour at room temperature. Solutions in wells were removed by pipetting, and each well was washed with 200 μL of a washing buffer [0.05% Tween 20-containing phosphate buffer (1 mM KH₂PO₄, 3 mM Na₂HPO.7H₂O, 154 mM NaCl pH7.4)] three times.

Each well was added with 100 μL of an Eu-N1-labeled mouse IgG antibody [available from PerkinElmer, 0.05 μg/mL 0.1% BSA-containing phosphate buffer (1 mM KH₂PO₄, 3 mM Na₂HPO.7H₂O, 154 mM NaCl pH 7.4) solution], and left still at room temperature for 1 hour, and then solutions in the wells were removed by pipetting, and each well was washed with 200 μL of a washing buffer [0.05% Tween 20-containing phosphate buffer (1 mM KH₂PO₄, 3 mM Na₂HPO.7H₂O, 154 mM NaCl pH 7.4)] three times.

Each well was added with 150 μL of Enhancement Solution (available from PerkinElmer), stirred for 5 minutes at room temperature, and left still for 15 minutes at room temperature, and then fluorescence was measured at excitation 340 nm/fluorescence 612 nm.

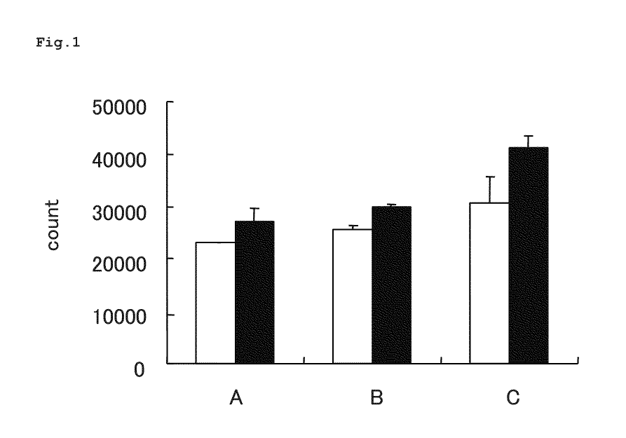

The result is shown in FIG. 1. Methylation frequency or an index value correlated therewith was high in the Solution C that simultaneously detects a plurality of target regions to be detected, compared to the Solution A and Solution B in which the target region to be detected is one kind. The index value correlated with methylation frequency of the target region to be detected was higher in genomic DNA extracted from Capan-2 cancer cell strain, than in human genomic DNA derived from a noncancerous cell.

In the present experiment, by simultaneously measuring methylation frequencies or index values correlated therewith of a plurality of target DNA regions, it is possible to measure methylation frequency or an index value correlated therewith more effectively than measuring methylation frequency or an index value correlated therewith of one target DNA region. Also, the higher methylation frequency or the index value correlated therewith in genomic DNA derived from Capan-2 cell strain which is a pancreatic cancer cell than in genomic DNA derived from a noncancerous tissue reveals that measurement of methylation frequency or an index value correlated therewith by mixing the target DNA regions used in the present examination will be a diagnostic index of cancer. Also it was confirmed that mixing of a specific oligonucleotide and a counter oligonucleotide, and the specific oligonucleotides utilized in the present examination is useful for cancer diagnosis.

### INDUSTRIAL APPLICABILITY

Based on the present invention, it becomes possible to provide a method of quantifying or detecting methylated DNA in a target DNA region in a genomic DNA contained in a biological specimen in a simple and convenient manner, and so on.
Free Text in Sequence Listing
SEQ ID NOs:13 to 24
Designed biotinated oligonucleotide for fixation
SEQ ID NOs:25 to 118
Designed oligonucleotide

1. A method of quantifying or detecting methylated DNA in a target DNA region possessed by genomic DNA contained in a biological specimen, comprising:
First step of separating double-stranded DNA derived from genomic DNA comprising the target DNA region contained in the biological specimen into single-stranded DNA;
Second step of mixing the single-stranded DNA, a methylated DNA antibody, and a specific oligonucleotide comprising a nucleotide sequence of a part of a complementary sequence to any of the following nucleotide sequences and not inhibiting binding between the methylated target DNA region in the single-stranded DNA and the methylated DNA antibody, to form a complex of the single-stranded DNA, the methylated DNA antibody, and the specific oligonucleotide; and
Third step of detecting or quantifying the methylated DNA in the target DNA region contained in the biological specimen by conducting detection or quantification depending on an identification function that is possessed by the methylated DNA antibody contained in the complex and available for detection:
(1) the nucleotide sequence of SEQ ID NO:1 or a nucleotide sequence having 80% or more sequence identity to the sequence,
(2) the nucleotide sequence of SEQ ID NO:2 or a nucleotide sequence having 80% or more sequence identity to the sequence,
(3) the nucleotide sequence of SEQ ID NO:3 or a nucleotide sequence having 80% or more sequence identity to the sequence,
(4) the nucleotide sequence of SEQ ID NO:4 or a nucleotide sequence having 80% or more sequence identity to the sequence,
(5) the nucleotide sequence of SEQ ID NO:5 or a nucleotide sequence having 80% or more sequence identity to the sequence,
(6) the nucleotide sequence of SEQ ID NO:6 or a nucleotide sequence having 80% or more sequence identity to the sequence,
(7) the nucleotide sequence of SEQ ID NO:7 or a nucleotide sequence having 80% or more sequence identity to the sequence,
(8) the nucleotide sequence of SEQ ID NO:8 or a nucleotide sequence having 80% or more sequence identity to the sequence,
(9) the nucleotide sequence of SEQ ID NO:9 or a nucleotide sequence having 80% or more sequence identity to the sequence,
(10) the nucleotide sequence of SEQ ID NO:10 or a nucleotide sequence having 80% or more sequence identity to the sequence,
(11) the nucleotide sequence of SEQ ID NO:11 or a nucleotide sequence having 80% or more sequence identity to the sequence,
(12) the nucleotide sequence of SEQ ID NO:12 or a nucleotide sequence having 80% or more sequence identity to the sequence,
(13) the complementary sequence to the nucleotide sequence of SEQ ID NO:1 or a nucleotide sequence having 80% or more sequence identity to the complementary sequence,
(11) the complementary sequence to the nucleotide sequence of SEQ ID NO:2 or a nucleotide sequence having 80% or more sequence identity to the complementary sequence,
(15) the complementary sequence to the nucleotide sequence of SEQ ID NO:3 or a nucleotide sequence having 80% or more sequence identity to the complementary sequence,
(16) the complementary sequence to the nucleotide sequence of SEQ ID NO:4 or a nucleotide sequence having 80% or more sequence identity to the complementary sequence,
(17) the complementary sequence to the nucleotide sequence of SEQ ID NO:5 or a nucleotide sequence having 80% or more sequence identity to the complementary sequence,
(18) the complementary sequence to the nucleotide sequence of SEQ ID NO:6 or a nucleotide sequence having 80% or more sequence identity to the complementary sequence,
(19) the complementary sequence to the nucleotide sequence of SEQ ID NO:7 or a nucleotide sequence having 80% or more sequence identity to the complementary sequence,
(20) the complementary sequence to the nucleotide sequence of SEQ ID NO:8 or a nucleotide sequence having 80% or more sequence identity to the complementary sequence,
(21) the complementary sequence to the nucleotide sequence of SEQ ID NO:9 or a nucleotide sequence having 80% or more sequence identity to the complementary sequence,
(22) the complementary sequence to the nucleotide sequence of SEQ ID NO:10 or a nucleotide sequence having 80% or more sequence identity to the complementary sequence,
(23) the complementary sequence to the nucleotide sequence of SEQ ID NO:11 or a nucleotide sequence having 80% or more sequence identity to the complementary sequence, and
(24) the complementary sequence to the nucleotide sequence of SEQ ID NO:12 or a nucleotide sequence having 80% or more sequence identity to the complementary sequence.

2. The method according to claim 1, wherein the specific oligonucleotide comprises any of the following nucleotide sequences:
(1) the nucleotide sequence of SEQ ID NO:13,
(2) the nucleotide sequence of SEQ ID NO:14,
(3) the nucleotide sequence of SEQ ID NO:15,
(4) the nucleotide sequence of SEQ ID NO:16,
(5) the nucleotide sequence of SEQ ID NO:17,
(6) the nucleotide sequence of SEQ ID NO:18,
(7) the nucleotide sequence of SEQ ID NO:19,
(8) the nucleotide sequence of SEQ ID NO:20,
(9) the nucleotide sequence of SEQ ID NO:21,
(10) the nucleotide sequence of SEQ ID NO:22,
(11) the nucleotide sequence of SEQ ID NO:23
(12) the nucleotide sequence of SEQ ID NO:24,
(13) the complementary sequence to the nucleotide sequence of SEQ ID NO:13,
(14) the complementary sequence to the nucleotide sequence of SEQ ID NO:14,
(15) the complementary sequence to the nucleotide sequence of SEQ ID NO:15,
(16) the complementary sequence to the nucleotide sequence of SEQ ID NO:16,
(17) the complementary sequence to the nucleotide sequence of SEQ ID NO:17,
(18) the complementary sequence to the nucleotide sequence of SEQ ID NO:18,
(19) the complementary sequence to the nucleotide sequence of SEQ ID NO:19,
(20) the complementary sequence to the nucleotide sequence of SEQ ID NO:20,
(21) the complementary sequence to the nucleotide sequence of SEQ ID NO:21,
(22) the complementary sequence to the nucleotide sequence of SEQ ID NO:22,
(23) the complementary sequence to the nucleotide sequence of SEQ ID NO:23, and
(24) the complementary sequence to the nucleotide sequence of SEQ ID NO:24.

3. The method according to claim 1, wherein a counter oligonucleotide is added in forming the complex in the Second step.

4. The method according to claim 1, wherein formation of the complex in the Second step is conducted in a reaction system containing a divalent cation.

5. The method according to claim 1, wherein the methylated DNA antibody in the complex formed in the Second step has been made to bind to a support before the Third step.

6. The method according to claim 1, further comprising, after the First step and before the Third step, the step of digesting unmethylated single-stranded DNA with a methylation sensitive restriction enzyme capable of digesting single-stranded DNA.

7. The method according to claim 1, further comprising, after the First step and before the Third step, the step of digesting unmethylated single-stranded DNA with a methylation-sensitive restriction enzyme in a reaction system containing a specific masking oligonucleotide comprising a recognition sequence for the methylation sensitive restriction enzyme as a part thereof.

8. The method according to claim 6, wherein the methylation sensitive restriction enzyme capable of digesting single-stranded DNA is HhaI.

9. The method according to claim 1, wherein the methylated DNA antibody is a methylcytosine antibody.

10. The method according to claim 1, wherein the biological specimen is mammalian blood, serum, plasma, bodily fluid, cell lysate or tissue lysate.

11. The method according to claim 1, wherein the DNA sample derived from genomic DNA contained in the biological specimen is digested in advance with a restriction enzyme recognition cleaving site for which is not present in the target DNA region of the genomic DNA.

12. The method according to claim 1, wherein the DNA sample derived from genomic DNA contained in the biological specimen has been digested with a methylation sensitive restriction enzyme.

13. The method according to claim 1, wherein the DNA sample derived from genomic DNA contained in the biological specimen has been digested with a methylation sensitive restriction enzyme in a reaction system containing a specific masking oligonucleotide.

14. The method according to claim 7, wherein the methylation sensitive restriction enzyme is HpaII or HhaI.

15. The method according to claim 1, wherein the DNA sample derived from genomic DNA contained in the biological specimen is a DNA sample purified in advance.

16. The method according to claim 1, wherein the target DNA region possessed by genomic DNA comprises a recognition cleaving site for the methylation sensitive restriction enzyme.

17. DNA comprising any of the following nucleotide sequences:
(1) the nucleotide sequence of SEQ ID NO:13,
(2) the nucleotide sequence of SEQ ID NO:14,
(3) the nucleotide sequence of SEQ ID NO:15,
(4) the nucleotide sequence of SEQ ID NO:16,
(5) the nucleotide sequence of SEQ ID NO:17,
(6) the nucleotide sequence of SEQ ID NO:18,
(7) the nucleotide sequence of SEQ ID NO:19,
(8) the nucleotide sequence of SEQ ID NO:20,
(9) the nucleotide sequence of SEQ ID NO:21,
(10) the nucleotide sequence of SEQ ID NO:22,
(11) the nucleotide sequence of SEQ ID NO:23
(12) the nucleotide sequence of SEQ ID NO:24,
(13) the complementary sequence to the nucleotide sequence of SEQ ID NO:13,
(14) the complementary sequence to the nucleotide sequence of SEQ ID NO:14,
(15) the complementary sequence to the nucleotide sequence of SEQ ID NO:15,
(16) the complementary sequence to the nucleotide sequence of SEQ ID NO:16,
(17) the complementary sequence to the nucleotide sequence of SEQ ID NO:17,
(18) the complementary sequence to the nucleotide sequence of SEQ ID NO:18,
(19) the complementary sequence to the nucleotide sequence of SEQ ID NO:19,
(20) the complementary sequence to the nucleotide sequence of SEQ ID NO:20,
(21) the complementary sequence to the nucleotide sequence of SEQ ID NO:21,
(22) the complementary sequence to the nucleotide sequence of SEQ ID NO:22,
(23) the complementary sequence to the nucleotide sequence of SEQ ID NO:23, and
(24) the complementary sequence to the nucleotide sequence of SEQ ID NO:24.
